(19) United States
(12) Patent Application Publication
(10) Pub. No.: US 2005/0143717 A1
(43) Pub. Date: Jun. 30, 2005

(54) Method of treatment of refractive errors using subepithelial or intrastromal corneal inlay with bonding coating

(76) Inventor: Gholam A. Peyman, New Orleans, LA (US)

Correspondence Address: BELL, BOYD, & LLOYD LLC, P. O. BOX 1135, CHICAGO, IL, 60690-1135, US

(21) Appl. No.: 10/784,169

(22) Filed: Feb. 24, 2004

Related U.S. Application Data
(63) Continuation-in-part of application No. 10/406,558, filed on Apr. 4, 2003.
(63) Continuation-in-part of application No. 10/356,730, filed on Feb. 3, 2003.
Application No. 10/356,730 is a continuation-in-part of application No. 09/843,141, filed on Apr. 27, 2001, now Pat. No. 6,551,307.
(60) Provisional application No. 60/449,617, filed on Feb. 26, 2003.

(51) Int. Cl. A61B 18/20

(52) U.S. Cl. 606/5

(57) ABSTRACT

A method of treatment of refractive errors of an eye, the eye including a central visual axis and a cornea with a first corneal layer overlying a second corneal layer, comprising the steps of separating a first surface of the first corneal layer from a second surface of the second corneal layer, thereby forming a flap and exposing the second surface, implanting on the second surface an inlay adapted to correct a refractive error of the eye, coating a surface of the inlay with a compound that promotes bonding with the cornea, and replacing the flap over the inlay.

### RELATED APPLICATIONS

This application is a continuation-in-part of U.S. patent application Ser. No. 10/406,558, filed Apr. 4, 2003 which claims the benefit of U.S. Provisional Application Ser. No. 60/449,617, filed Feb. 26, 2003, and is a continuation-in-part of U.S. patent application Ser. No. 10/356,730, filed Feb. 3, 2002 which is a continuation-in-part of U.S. patent application Ser. No. 09/843,141, filed Apr. 27, 2001, the entire contents of each of which are herein incorporated by reference.

### FIELD OF THE INVENTION

The present invention relates to a method for treating refractive errors of a patient's eye. More specifically, an inlay is selected for correcting the patient's refractive error, implanted, and immobilized in proper position on the patient's cornea using a bonding compound, such as an organic coating.

### BACKGROUND OF THE INVENTION

Conventional methods of treating refractive errors involve implanting a corrective lens by removing a portion or flap of one layer of the patient's cornea, such as the epithelium, implanting the lens on a second layer below the epithelium, and then waiting for the removed flap of the epithelium to grow back. Conventional methods also involve applying a material on the lens prior to implantation that promotes growth of the epithelium.

Presbyopia, which is blurred vision of close up objects, e.g. when reading, typically occurs due to aging of the eye. A conventional method for correcting the refractive error in a cornea is keratophakia, i.e., implantation of a lens inside the cornea. Keratophakia uses an implant which is placed into the cornea approximately equidistant from the exterior surface of the cornea and the interior surface. The procedure is usually done by first preparing a lens from corneal donor tissue or synthetic material using a cryo-lathe. The lens is implanted by removing a portion of the cornea with a device called a microkeratome, and the tissue is sutured back into place over the lens. However, there can be problems when microkeratomies are used for cutting the cornea. First, irregular keratectomies or perforations of the eye can result. Second, the recovery of vision can be rather prolonged.

Another surgical technique exists that uses a femtosecond laser to separate layers inside the stromal, at least two-thirds of the distance from the top surface of the cornea to the inside of the eye. An incision is made to access this area and a solid inlay is inserted to help correct myopia in the eye. By separating the layers in the bottom two-thirds of the stromal, it is difficult to access the separated area to insert the inlay and virtually impossible to change or modify the inlay without another extensive surgical procedure. This procedure requires making an incision which is parallel to the visual axis and is limited in the lateral direction by a maximum size of 0.3 mm to encase a relatively rigid inlay that forces the tissue in the lateral direction.

Additional surgical techniques exist that use ultraviolet light and short wavelength lasers to modify the shape of the cornea. For example, excimer lasers, such as those described in U.S. Pat. No. 4,840,175 to Peyman, which emit pulsed ultraviolet radiation, can be used to decompose or photoablate tissue in the live cornea so as to reshape the cornea.

Specifically, the Peyman patent discloses the laser surgical technique known as laser in situ keratomycosis (LASIK). In this technique, a portion of the front of the live cornea can be cut away in the form of a flap having a thickness of about 160 microns. This cut portion is removed from the live cornea to expose an inner surface of the cornea. A laser beam is then directed onto the exposed inner surface to ablate a desired amount of the inner surface up to 150-180 microns deep. The cut portion is reattached over the ablated portion of the cornea and assumes a shape conforming to that of the ablated portion. Additionally, in the Lasik procedure, a femtosecond laser can be used to cut and separate the flap.

Other conventional methods that have been employed specifically to correct presbyopia have been unsuccessful. Some of those methods include using an excimer laser to ablate the peripheral part of the cornea, expanding the sclera behind the limbus area of the cornea, implanting a plus lens inside the corneal stroma, using a multifocal intraocular lens after removal of the cataractous lens, bifocal glasses and bifocal contact lenses.

However, because only certain amount of cornea can be ablated without the remaining cornea becoming unstable or experiencing outbulging (ectasia), this technique is not especially effective in correcting very high myopia. That is, a typical cornea is on average about 500 microns thick. The laser ablation technique requires that at least about 250 microns of the corneal stroma remain after the ablation is completed so that instability and outbulging do not occur. Also, these conventional implants, while correcting a refractive error of the patient, also distort the normal vision of the patient.

Additional methods for correcting the refractive error in the eye include inserting an implant in-between layers of the cornea. Generally, this is achieved using several different methods. The first method involves inserting a ring between layers of the cornea, as described in U.S. Pat. No. 5,405,384 to Silvestrini. Typically, a dissector is inserted in the cornea and forms a channel therein. Once it is removed, a ring is then inserted into the channel to alter the curvature of the cornea. In the second method, a flap can be created similarly to the LASIK procedure and a lens can be inserted under the flap, as described in U.S. Pat. No. 6,102,946 to Nigam. The third method involves forming a pocket using an instrument, and inserting an implant into the pocket, as described in U.S. Pat. No. 4,655,774 to Choyce.

However, with the above described techniques, a knife or other mechanical instrument is generally used to form the channel, flap or pocket. Use of these instruments may result in damage or imprecision in the cut or formation of the desired area in which the implant is placed. Additionally, these conventional techniques do not include determination and testing of an appropriate implant for correcting a refractive error of a particular patient.

Prior methods for the treatment of presbyopia have been unsuccessful. One prior method involved implantation of a disc shaped inlay or lens over the central visual axis of the cornea. The disc inlay had a high index of refraction to correct presbyopia and/or hyperopia. However, because the disc covered the center area around the visual axis, the patient's farsighted vision was blurred by the inlay. Another prior method involved a ring shaped inlay implanted around the visual axis. The ring inlay had a lower index of refraction or an index of refraction that is the same as the cornea and therefor corrected myopic refractive errors instead of hyperopic or presbyopic error.

Therefore, there exists a need for an inlay and improved method of correcting refractive error that preserves the corneal flap and immobilizes the inlay in its proper position during the implantation process.

### SUMMARY IF THE INVENTION

Accordingly, it is an object of the present invention to provide an improved method for modifying the cornea of an eye, particularly for correcting presbyopia.

Another object of the present invention is to provide a method for modifying the cornea of an eye that results in a precise separation between layers in the cornea.

Still another object of the present invention is to provide a method for modifying the cornea of an eye that allows for corrective measures that avoid or eliminate outbulging or instability in the cornea.

Yet another object of the present invention is to provide a method for modifying the cornea of an eye that avoids or eliminates most of the risks of damage due to use of knives or other mechanical instruments.

Another object of the present invention is to provide a method for treating a refractive error of the cornea by implanting a corrective inlay under the epithelium.

Still another object of the present invention is to provide a device for removing the epithelium to form a flap allowing an inlay to be implanted without damaging the epithelium.

Another object of the present invention is to provide an inlay that corrects presbyopia without distorting farsighted vision.

Yet another object of the present invention is to provide a method for selecting the appropriate inlay to correct a refractive error, such as presbyopia.

Still another object of the present invention is to provide a method for treating refractive errors that preserves the epithelium flap and immobilizes the corrective inlay in proper position with respect to the patient's visual axis.

The foregoing objects are basically attained by a method of treatment of refractive errors of an eye, the eye including a central visual axis and a cornea with a first corneal layer overlying a second corneal layer, comprising the steps of separating a first surface of the first corneal layer from a second surface of the second corneal layer, thereby forming a flap and exposing the second surface, implanting on the second surface an inlay adapted to correct a refractive error of the eye, coating a surface of the inlay with a compound that promotes bonding with the cornea, and replacing the flap over the inlay.

The foregoing objects are also attained by a method of treatment of refractive errors of an eye, the eye including a central visual axis and a cornea with a first corneal layer overlying a second corneal layer, comprising the steps of separating a first surface of the first corneal layer from a second surface of the second corneal layer, thereby exposing the second surface, implanting on the second surface an inlay adapted to correct a refractive error of the eye, coating a surface of the inlay after implanting the inlay with a compound that promotes bonding with the cornea, coating the exposed second surface adjacent the inlay with the compound, and drying the compound coating the inlay and the exposed second surface, thereby forming a drape over the inlay and bonding the inlay to the second surface.

Other objects, advantages, and salient features of the present invention will become apparent to those skilled in the art from the following detailed description, which, taken in conjunction with the annexed drawings, discloses preferred embodiments of the invention.

### BRIEF DESCRIPTION OF THE DRAWINGS

Referring to the drawings which form a part of this disclosure:

FIG. 1 illustrates a method of forming a pocket in the cornea of an eye, by irradiating the cornea with an ultrashort pulse laser, according to the preferred embodiment of the present invention;

FIG. 2 is an elevational front view of the eye and the pocket of FIG. 1;

FIG. 3 is an elevational front view of a second embodiment of the invention wherein two pockets are formed by an ultrashort pulse laser;

FIG. 4 is an elevational front view of a third embodiment of the present invention wherein four pockets are formed by an ultrashort pulse laser;

FIG. 5 is an elevational front view of a fourth embodiment of the present invention wherein no central portion is left attached in a pocket formed by the ultrashort pulse laser;

FIG. 6 is an elevational front view of a fifth embodiment of the present invention wherein a needle is used to inject ocular material into a pocket formed by an ultrashort pulse laser;

FIG. 7 is a cross-sectional side view of the eye of FIG. 6 with a contact lens placed on the external surface of the cornea to shape the ocular material;

FIG. 8 is a cross-sectional side view of a eye having a ring-shaped pocket formed in between layers of the cornea with a contact lens placed on the external surface of the cornea to shape the ocular material;

FIG. 9 is a front elevational view of a split ring ocular implant for use in the procedure shown in FIGS. 1-4 and 19-24;

FIG. 10 is a front elevational view of a two part ocular implant for use in the procedure shown in FIGS. 1-4 and 19-24;

FIG. 11 is a front elevational view of a three part ocular implant for use in the procedure shown in FIGS. 1-4 and 19-24;

FIG. 12 is a side elevational view in cross-section of the ocular implant of FIG. 9, taken along lines 12-12;

FIG. 13 is a side elevational view in cross-section of the ocular implant of FIG. 10, taken along lines 13-13;

FIG. 14 is a side elevational view in cross-section of an arcuate ocular implant for use in the procedure shown in FIGS. 1-4 and 19-24;

FIG. 15 is a side elevational view in cross-section of multiple ocular implants stacked on top of one another for use in the procedure shown in FIGS. 1-4 and 19-24;

FIG. 16 is a side elevational view in cross-section of an ocular implant having a non-uniform thickness for use in the procedure shown in FIGS. 1-4 and 19-24;

FIG. 17 is a front elevational view in cross-section of an ocular implant having four separate portions for use in the procedure shown in FIGS. 1-4 and 19-24;

FIG. 18 is a front elevational view in cross-section of an ocular implant having two portions of different thickness for use in the procedure shown in FIGS. 1-4 and 19-24;

FIG. 19 is a side elevational view in cross section similar to that shown in FIG. 1 with the incision in the pocket open;

FIG. 20 is a side elevational view in cross section similar to that shown in FIG. 19, except that an annular or circular ocular implant has been introduced through the incision and between the internal surfaces;

FIG. 21 is a side elevational view in cross section of a probe irradiating a portion of the ocular material to reduce the volume of the portion;

FIG. 22 is a side elevational view in cross section of a probe irradiating a portion of the ocular material to increase the volume of the portion;

FIG. 23 is a side elevational view in cross section similar to that shown in FIG. 19, except that a portion of the external surface of the cornea has been ablated by a laser;

FIG. 24 is a side elevational view in cross section of the cornea with a flap formed thereon and a laser ablating a portion of the ocular material;

FIG. 25 is a side elevational view in cross section of an eye similar to that shown in FIG. 20, except that a flap has been formed on the surface of the cornea.

FIG. 26 is a side elevational view in cross section of the eye of FIG. 25, with the flap moved to expose an internal corneal surface;

FIG. 27 a side elevational view in cross section of the eye of FIG. 26, with a laser ablating a portion of the exposed internal corneal surface;

FIG. 28 is a side elevational view in cross section of the eye of FIG. 27, with the flap replaced over the ablated internal corneal surface;

FIG. 29 is a top perspective view of a device for forming the flap of FIGS. 25-28;

FIG. 30 is a top perspective view of a suction device for removing the flap of FIGS. 25-28;

FIG. 31 is a method of forming a flap in the cornea of an eye, by cutting the cornea using a cutting tool;

FIG. 32 is a plan view of a semi-ring shaped inlay in accordance with an embodiment of the present invention, showing the inlay being implanted in the cornea underneath the epithelium;

FIG. 33 is an exploded side elevational view of the inlay illustrated in FIG. 32, showing the inlay being implanted on a corneal surface and under an epithelial flap;

FIG. 34 is a side elevational view taken in section along line 34-34 of FIG. 32;

FIG. 35 is a side elevation view similar to FIG. 34, showing the use of an intraocular lens with the inlay;

FIG. 36 is a side elevational view of the inlay illustrated in FIG. 32, showing the inlay implanted on the corneal surface with the epithelial flap removed and the use of a laser with the inlay;

FIG. 37 is a side elevational view of an inlay in accordance with an embodiment of the present invention, showing the inlay having multiple layers and implanted under the epithelium;

FIG. 38 is a plan view of a ring-shaped inlay in accordance with an embodiment of the present invention, showing the inlay implanted in the cornea underneath the epithelium;

FIG. 39 is a side elevational view taken in section along line 39-39 of FIG. 38;

FIG. 40 is a plan view of a semi-ring shaped inlay formed of a plurality of segments in accordance with an embodiment of the present invention, showing the inlay implanted in the cornea underneath the epithelium;

FIG. 41 is a side elevational view taken in section along line 41-41 of FIG. 40;

FIG. 42 is a plan view of a ring-shaped inlay formed of a plurality of segments in accordance with an embodiment of the present invention, showing the inlay implanted in the cornea underneath the epithelium;

FIG. 43 is a side elevational view taken in section along line 43-43 of FIG. 42;

FIG. 44 is a plan view of an inlay including two separate sections in accordance with an embodiment of the present invention, showing the inlay implanted in the cornea underneath the epithelium;

FIG. 45 is a side elevational view taken in section along line 45-45 of FIG. 44;

FIG. 46 is a plan view of an inlay including two overlapping sections in accordance with an embodiment of the present invention, showing the inlay implanted in the cornea underneath the epithelium;

FIG. 47 is a side elevational view taken in section along line 47-47 of FIG. 46;

FIG. 48 is a plan view of a rectangular inlay in accordance with an embodiment of the present invention, showing the inlay implanted in the cornea underneath the epithelium;

FIG. 49 is a side elevational view taken in section along line 49-49 of FIG. 48;

FIG. 50 is a plan view of a circular inlay in accordance with an embodiment of the present invention, showing the inlay implanted in the cornea underneath the epithelium;

FIG. 51 is a side elevational view taken in section along line 51-51 of FIG. 50;

FIG. 52 is a plan view of an inlay formed of a row of segments in accordance with an embodiment of the present invention, showing the inlay implanted in the cornea underneath the epithelium;

FIG. 53 is a side elevational view taken in section along line 53-53 of FIG. 52;

FIG. 54 is a plan view of an inlay formed of multiple rings in accordance with an embodiment of the present invention, showing the inlay implanted in the cornea underneath the epithelium;

FIG. 55 is a side elevational view taken in section along line 55-55 of FIG. 54;

FIG. 56 is a side elevational view in partial section of a suction device in accordance with the present invention, showing the suction device on the epithelium of the cornea prior to separation of an epithelial flap;

FIG. 57 is a side elevational view in partial section of the suction device illustrated in FIG. 56, showing the epithelial flap removed from the corneal surface by the suction device;

FIG. 58 is a plan view of the cornea illustrated in FIG. 56, showing markings on the cornea;

FIG. 59 is a top plan view of the suction device illustrated in FIG. 56;

FIG. 60 is a bottom plan view of the suction device illustrated in FIG. 56;

FIG. 61 is a side elevational view in partial section of an alternative suction device in accordance with the present invention, showing the suction device on the epithelium of the cornea prior to separation of an epithelial flap;

FIG. 62 is a side elevational view in partial section of the suction device illustrated in FIG. 61, showing the epithelial flap removed from the corneal surface by the suction device;

FIG. 63 is a bottom plan view of the suction device illustrated in FIG. 61;

FIG. 64 is a top plan view of the suction device illustrated in FIG. 61;

FIG. 65 is a side elevational view in section of an alternative suction device in accordance with the present invention, showing the suction device on the cornea prior to separation of a flap;

FIG. 66 is a top plan view of a plate of the suction device illustrated in FIG. 65;

FIG. 67 is a top plan view of an alternative plate for the suction device illustrated in FIG. 65;

FIG. 68 is a top plan view of an exemplary inlay in accordance with the present invention, showing a blend zone of the inlay;

FIG. 69 is a top plan view of a lens in accordance with the present invention, showing the lens supporting an exemplary inlay that is semi-ring shaped;

FIG. 70 is a top plan view similar to FIG. 69 of a lens in accordance with the present invention, showing the lens supporting an exemplary inlay that is smaller than the inlay of FIG. 69 and is semi-ring shaped;

FIG. 71 is a top plan view similar to FIG. 69 of a lens in accordance with the present invention, showing the lens supporting an exemplary inlay that is larger that the inlay of FIG. 69 and is semi-ring shaped;

FIG. 72 is a top plan view of a lens in accordance with the present invention, showing the lens supporting an exemplary inlay that is ring shaped;

FIG. 73 is a top plan view similar to FIG. 72 of a lens in accordance with the present invention, showing the lens supporting an exemplary inlay that is smaller that the inlay of FIG. 72 and is ring shaped;

FIG. 74 is a top plan view similar to FIG. 72 of a lens in accordance with the present invention, showing the lens supporting an exemplary inlay that is larger that the inlay of FIG. 72 and is ring shaped;

FIG. 75 is a top view of a lens in accordance with the present invention, showing the lens supporting an exemplary inlay that is substantially semi-circular in shape;

FIG. 76 is a top plan view of a lens in accordance with the present invention, showing the lens supporting an exemplary inlay that is substantially triangular in shape;

FIG. 77 is a top plan view of a lens in accordance with the present invention, showing the lens supporting an exemplary inlay that includes multiple rings;

FIG. 78 is a top plan view of a lens in accordance with the present invention, showing the lens supporting multiple exemplary inlays that are semi-ring shaped;

FIG. 79 is a top plan view of a lens in accordance with the present invention, showing markings on the lens;

FIG. 80 is a side elevational view in section of a lens in accordance with the present invention, showing the lens supporting an exemplary inlay in a recess of the lens;

FIG. 81 is a side elevational view in section of an inlay in accordance with the present invention, showing the lens supporting the inlay between two layers of the lens;

FIG. 82 is a side elevational view in section of an inlay in accordance with the present invention, showing the lens supporting the inlay on an outer surface of the lens;

FIG. 83 is an exploded side elevational view taken in section showing the flap of the patient's cornea being separated and lifted and the corrective inlay being implanted in accordance with the present invention;

FIG. 84 is a side elevational view taken in section similar to FIG. 83, showing the corrective inlay illustrated in FIG. 83 positioned in the patient's cornea;

FIG. 85 is a side elevational view taken in section similar to FIG. 84, showing the application of a liquid coating on the corrective inlay;

FIG. 86 is a side elevational view taken in section similar to FIG. 84, showing the drying of the coating on the corrective inlay;

FIG. 87 is a side elevational view taken in section similar to FIG. 84, showing the flap replaced over the inlay and the coating dried on the inlay; and

FIG. 88 is an enlarged side elevational view in section of the corrective inlay illustrated in FIG. 83, showing a coating applied before implantation and substantially enclosing the inlay.

### DETAILED DESCRIPTION OF THE PREFERRED EMBODIMENT

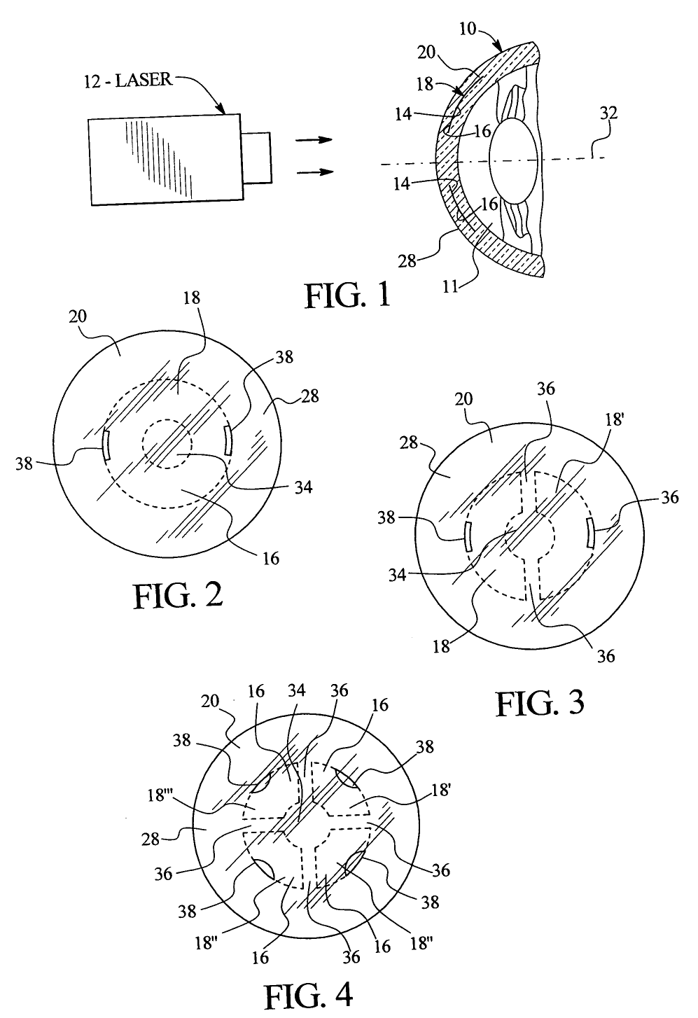

As initially shown in FIGS. 1, 2 and 19-24, the refractive properties of eye 10 can be altered by using laser 12 to separate an inner portion of the cornea into first internal corneal surface 14 and second internal corneal surface 16, creating internal corneal pocket 18 in the cornea 20 and then placing ocular material or an implant 22 in the pocket 18. Additionally, the cornea can be shaped by using a second laser 24 to ablate a portion 26 of the surface 28 of the cornea 16, or an external lens 29 to mold the ocular material.

To begin, the refractive error in the eye is measured using wavefront technology, as is known to one of ordinary skill in the art. For a more complete description of wavefront technology see U.S. Pat. No. 6,086,204 to Magnate, the entire contents of which is incorporated herein by reference. The refractive error measurements are transmitted to a computerized lathe (not shown) or other lens-shaping machine, where the shape of ocular material is determined using the information from the wavefront device. Alternatively, the ocular material 22 can be manufactured or shaped prior to the use of the wavefront technology and can be stored in a sterilized manner until that specific shape or size is needed.

Ocular material or inlay 22 has a first surface 21 and a second surface 23 and is porous to allow oxygen and nutrients to pass therethrough. Materials that are suitable for these purposes are preferably any polymer or hydrogel having about 50% water content; however, the water content can be any percentage desired. The ocular material may be formed from synthetic or organic material or a combination thereof. For example, the ocular material can be collagen combined with or without cells; a mixture of synthetic material and corneal stromal cells; silicone or silicone mixed with collagen; mucopolysacharide; chodrotin sulfate; elsatins; methylmetacrylate; hydrogel; any transparent material, such as polyprolidine, polyvinylpylidine, polyethylenoxide, etc.; or any deformable and/or porous polymer, which can change its shape with radiation after implantation. The collagen can be a semiliquid, a gel, human or other animal, or it can de derivatized.

Generally, ocular material 22 is preferably about 0.5 mm to 5 mm wide. The thickness is preferably about 5-2000 microns, and more preferably less than 200 microns. The inside edge can be thinner or thicker than the outside edge; for example, the inside edge can have a thickness of about 1-100 microns, while the outside edge has a thickness of about 20-3000 microns. However, the ocular material can have any thickness or configuration that would allow it to elevate or move any portion of surface 14 relative to surface 16. The thickness and position of ocular material 22 generally defines the degree of correction.

Preferably, ocular material 22 is a liquid or a gel that can be injected through the surface of the cornea using an injection device 25, such as a needle, without making a large incision or opening in the surface of the lens, as seen in FIG. 6. By injecting a gel into a pocket in this manner, the gel is confined to the corneal pocket 18 and will settle or move in the pocket in a predictable configuration or distribution. In other words, the gel will not flow through the layers of the cornea, but will rather stay inside the structure or confines of the pocket. The gel can be inserted into a pocket that encompasses the entire front of the cornea, or extend past the cornea and Bowman layer to the sclera. By extending the pocket past the Bowman layer, the portion of the cornea above the pocket would become loose. The injection of the gel would allow lifting of the Bowman layer, lifting up the entire front surface of the cornea, allowing the eye to be reshaped as desired. However, the gel can be injected or positioned into any size pocket desired and the pocket does not have to encompass the entire front of the cornea. Additionally, as described below, the ocular material does not necessarily need to be a gel in this process and may be a lens or any other desired material.

Furthermore, the ocular material 22 can include a silicone polymer which includes loose monomers that are responsive to light (both visible and invisible) within a certain wavelength range, such as the short ultraviolet wavelength range or the blue light wavelength range. In response to the light, the monomers become aggravated, and cross-linking occurs which increases the volume of the area of ocular material 22 or a portion of the ocular material, without substantially ablating the ocular material 22, as well as fixing or hardening the ocular material.

The ocular material 22 can also include a polymer comprising a polycarbonate or acrylic material containing a dye or dyes manufactured, for example, by Centex Company. The dye or dyes absorb light within a certain wavelength range, such as the infrared wavelength range, which causes slight melting or reduction of the material or a portion of the ocular material, as well as solidification. This melting or reduction results in a decrease or flattening of the irradiated area of the ocular material 22, and thus reduces the volume of that area for purposes discussed in more detail below, without substantially ablating the ocular material 22.

See also U.S. application Ser. No. 09/532,516, filed Mar. 21, 2000 which is herein incorporated by reference, for a further discussion of swelling or shrinking of ocular material.

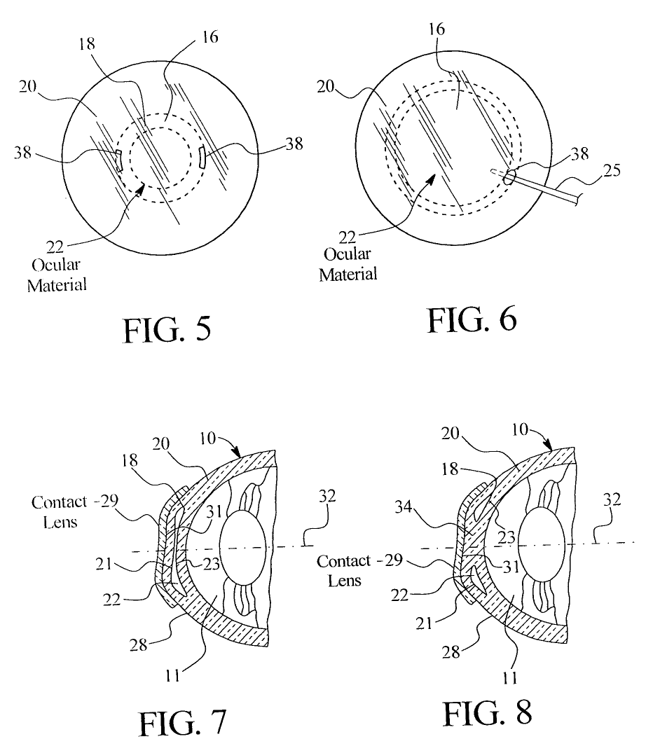

Ocular material 22 can also be a lens. When a lens, it can be any shape or sized desired. As seen in FIGS. 6-15, the lens is preferably substantially ring-shaped; but can be a circular or semicircular inlay. For example, unitary lenses 22a-c have a split 30 or have multiple portions that couple or fit together (FIGS. 9-11), lens 22b is flat (FIG. 13), lens 22d is arcuate (FIG. 14), and lens 22a has tapered edges (FIG. 12). Additionally, ocular material 22 may have any combination of these properties. When the lens has multiple portions, as seen in lenses 22f and 22g, the portions can couple together, simply abut one another, they can lay near each other, not necessarily touching each other or the lens portions can be separated from each other (FIGS. 17 and 18). Lens 22b can have multiple layers on top of each other (FIG. 15), or lens 22c and 22g can have two sides with different thickness (FIGS. 16 and 18), which would help to correct astigmatism. Additionally, the lens preferably allows light in the visible spectrum to pass therethrough and can have different or similar refractive properties to the refractive properties of the cornea, it can have pigmentation added thereto to change the color of the lens or it can be photochromatic. Furthermore, it is not necessary for the lens to have a hole or aperture therethrough. The lens can have a substantially planar surface or an arcuate surface with no holes or apertures therein, as seen specifically in FIG. 5.

As seen specifically in FIGS. 1-5, a laser 12 is aimed at an internal portion of the cornea, adjacent the external surface of the cornea of the eye and fired. Preferably, the laser is focused to create the pocket 18 in the first one-third of the cornea, and not in the back of the cornea. In other words, the pocket is preferably formed adjacent surface 28 or closer to surface 28 then to the interior or anterior chamber 11 of eye 10. By forming the pocket in the first one-third of the cornea, the pocket or pockets may extend beyond the Bowmans layer and the cornea, to create a large pocket, which would allow raising of the entire front portion of the cornea, as described above. The laser preferably separates an internal area of the cornea offset from the main optical or visual axis 32 into first 14 and second 16 substantially ring-shaped internal surfaces to form the circular or ring-shaped corneal pocket 18. First internal corneal surface 14 faces in a posterior direction of cornea 20 and the second internal corneal surface 16 faces in an anterior direction of the cornea 20. The distance from first internal corneal surface 14 to the exterior corneal surface 28 is preferably a uniform thickness of about 10-250 microns, and more preferably about 80-100 microns, but can be any suitable thickness and does not necessarily need to be substantially uniform. A portion 34 of first and second surfaces 14 and 16 preferably remains attached to each other by an area located at the main optical axis 32. However, the laser can form a pocket 18 of any suitable configuration, such as a pocket that is not attached at the main optical axis (FIG. 5), two substantially similar pockets 18 and 18′(FIG. 3) or four pockets 18, 18′, 18′ and 18′″ (FIG. 4). When multiple pockets are formed, preferably the pockets are separated by a portion 36, which is an area where first and second surfaces 14 and 16 remain attached. However, the pocket or pockets may be any number, shape or size desired and they do not need to be circular or ring-shaped. Furthermore, a flap similar to the above-described pocket, and as described in U.S. patent application Ser. No. 09/758,263 can be formed using laser 12 or a cutting tool or knife 90 (FIG. 31), such as a microkeratome, or any other device known in the art.

Laser 12 preferably is an ultrashort pulse laser, such as a femto, pico, or attosecond laser; but may be any light emitting device suitable for creating a pocket in the cornea as described above. The ultrashort pulse laser is positioned in front of the eye and is focused at the desired depth in the cornea and in the desired pocket configuration. Ultrashort pulse lasers are desired since they are high precision lasers that require less energy than conventional lasers to cut tissue and do not create “shock waves” that can damage surrounding structures. Cuts made by ultrashort pulse lasers can have very high surface quality with accuracy better than 10 microns, resulting in more precise cuts than those made with mechanical devices or other lasers. This type of accuracy results in less risks and complications than the procedures using other lasers or mechanical devices.

As seen in FIGS. 2-5, an incision or opening 38 is made in the surface 28 of the cornea to access pocket 18 or pockets 18′, 18″ and 18′″. Preferably, the incision 38 is made at the periphery of the pocket; however, it may be made anywhere desired that would allow access to the pocket 18. Additionally, multiple incisions can be made that would allow access to different portions of pocket 18 or different pockets 18′, 18″ and 18′″. A carved instrument (not shown) can be inserted through the incision, which would dissect the pocket, if needed. A carved instrument is generally used to extend the pocket 18 past the cornea or Bowmans layer to the sclera as described above. However, a large incision may not be necessary, as in the case where a gel is inserted using a needle, as described above.

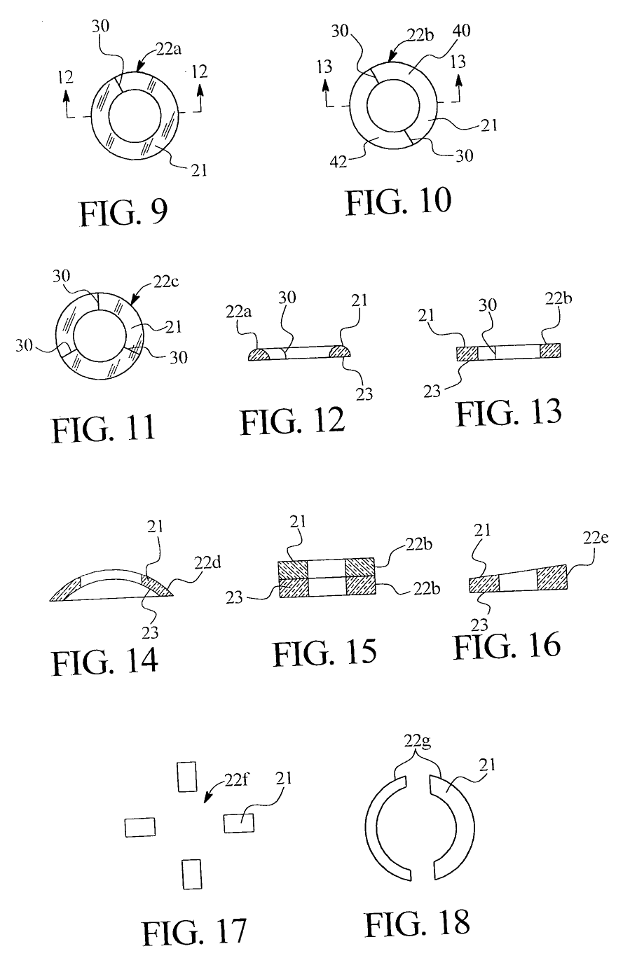

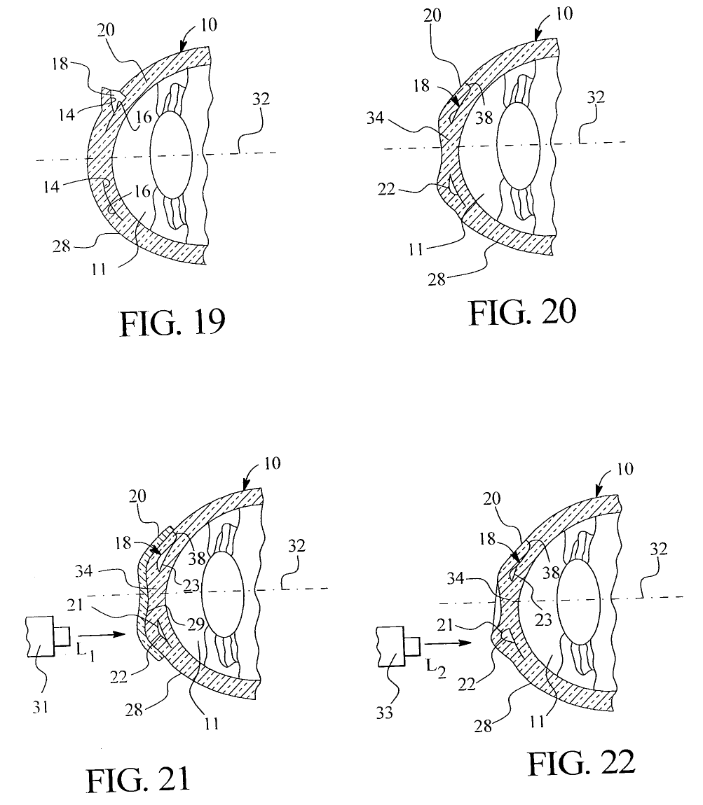

As seen in FIGS. 19 and 20, the ocular material 22 is then inserted through the incision 28 or any other opening by opening the incision using any device known in the art, such as spatula or microforceps or any other device. Preferably, when a lens is used, it has at least two separate portions 40 and 42 (FIG. 10) or has a split 30 (FIG. 9) that allow the ocular material 22 to be positioned or introduced around or at least partially encircling the main optical axis 32 or portion 34 and in between the first and second internal surfaces 14 and 16 that define the pocket 18. However, as stated above the first and second surfaces 14 and 16 do not necessarily have to be attached at the main optical axis and in such a case, ocular material 22 is merely placed in pocket 18.

As seen in FIGS. 7 and 8, when ocular material is injected or placed into pocket 18, an external contact lens 29 can be placed on the external surface of the cornea, which would allow the gel to be shaped or redistributed and, thus, the cornea to be reshaped in any manner desired. The proper size and shape of the contact lens 29 is determined by the information received from the wavefront technology. Lens 29 is preferably a temporary lens that would allow light if the visible spectrum to pass therethrough. The contact lens back surface 31 forces the gel to distribute evenly until the topographically desired configuration is achieved. Additionally, the opening 38 may allow a small amount of gel to escape, if needed, to adjust the shape and size of the ocular-material 22. Wave front technology can then be used to determine if the desired correction has been achieved, and if it has not the gel can be removed via an incision and the process repeated at a later time.

Once the ocular material is in place, the patient's eye can be monitored or measured and a laser, probe 31 or other heating device can be used to reduce the overall thickness of the ocular material 22, if necessary. For instance, the ocular material 22 can initially be about 500 microns thick for ease of handling. Then, once the material 22 is positioned in the pocket 18 of the cornea, in the manner described above, the probe 40 (i.e., infrared light) can be directed to material 22 so as to reduce the overall thickness of material 22, as desired. Hence, a 500 micron thick portion of the material can be reduced, for example, to about 100 microns or any suitable thickness by the heating device. It is noted that when the pulsed laser light is focused properly to a location within ocular material 22, it can disrupt and thus shrink or melt ocular material 22 without the need of an absorbent dye. An example of such a laser is an ultrashort pulse laser, which emits nano-second pulses, pico-second pulses or femto-second pulses of laser light. Furthermore, laser light having a wavelength that is absorbed by water, or other types of energy such as microwave radiation, radio frequency radiation, or thermal energy, can be used to cause shrinkage in the lens.

As shown in FIG. 21, an area of the material is irradiated with energy L₁, such as infrared light, laser light, microwave energy, radio frequency energy, or heat applied by a probe or laser 31, to cause the area of the lens to shrink or, in other words, reduce in volume. This shrinkage occurs without damage to the ocular material or other portion of the cornea 20. Accordingly, the shrinkage causes a change in the shape of the ocular material area, and thus changes the refractive power of the cornea 20 to further correct for the remaining vision disorder that was not fully corrected by the ocular material 22. The ocular material can be irradiated directly through the cornea or through lens 29.

Alternatively, the patient's vision can be monitored as the cornea 20 heals to determine if the size and shape of the ocular material 22 should be increased. The size or shape of the ocular material can be changed, and therefore the curvature of the cornea 20 can be changed without surgically opening the pocket 18. That is, as discussed above, the ocular material 22 can include certain monomers which, when irradiated with light within a certain wavelength range (e.g., blue or ultraviolet light), become agitated and cross-link, which causes the ocular material 22 to increase in size at the area of the irradiation.

As shown in FIG. 22, an area of ocular material 22 is irradiated by probe 33 or laser light L₂, which passes through the layer 21. The laser light L₂ has a wavelength, such as long ultraviolet wavelength or light within the blue light spectrum, to aggravate the monomers, which causes a cross-linking effect that increases the volume of the ocular material 22 in the area being irradiated. Hence, as the thickness of the ocular material 22 increases, this increase thickness changes the curvature of the cornea as shown, thus changing the refractive power of the cornea to a degree necessary to correct the remainder of the vision disorder that was not corrected by the insertion of the ocular material 22. The ocular material can be irradiated directly through the cornea or through lens 29.

Furthermore, a chemical can be used to polymerize or solidify the ocular material, when the ocular material is a collegen solution. Preferably, the chemical is applied to the external surface of the cornea and passes through the cornea and into the pocket 18, where it comes into contact with ocular material 22 and polymerizes the material. Preferably, the chemical used to polymerize the collegen solution is preferably about, 0.1 moler to 0.5 moler and more preferably about 0.2 moler to 0.4 moler of sodium persulphate diluted in a 0.02 moler phosphate buffer having a pH of about 8.0. However, the polymerizing chemical and the ocular material may be any suitable chemical and material known to one skilled in the art.

Furthermore, if necessary, the collegen solution can be depolymerized or returned to a gel or liquid state by applying glugaric anhydride in the same manner as described above for sodium persulphate. However, the depolymerization chemical can be any suitable chemical known in the art. Once the ocular material is depolymerized, the procedure can be repeated as often as desired. In other words, the refractive properties of the eye can be remeasured and reset and the material can be repolymerized as many times as desired until the correct refractive measurement is achieved.

To clean or wash the above chemicals from the eye, a disodium phosphate of about 0.02 molar and pH of 8.5 can be applied to the surface of the cornea.

Once the ocular material is in place and/or cross-linked or solidified as described above, the refractive properties of the eye can be remeasured using wavefront technology, and it can be determined if any refractive error remains in the eye. Generally, the refractive error is less than ±2.0 diopters sphere or astigmatism.

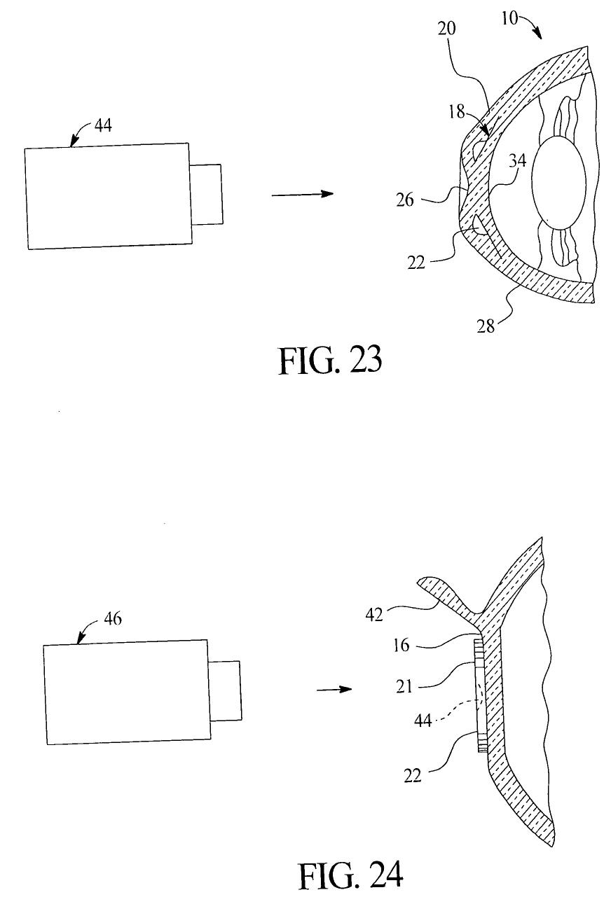

To reduce or eliminate this small refractive error, a second laser 44, preferably an excimer laser, can then be aimed and fired at the external surface of the cornea 24, ablating a portion 26 of the cornea, as seen in FIG. 23. Preferably, about 1-100 micron thickness is ablated, but any thickness that achieves the desired result can be ablated from the exterior surface of the cornea. The excimer laser can be applied either through the corneal epithelium or the epithelium can be reopened initially using diluted alcohol (less than 20% alcohol) or a brush. The second laser preferably ablates portion 26 of surface 22 that overlies the portion 34 attaches, but may ablate any portion desired.

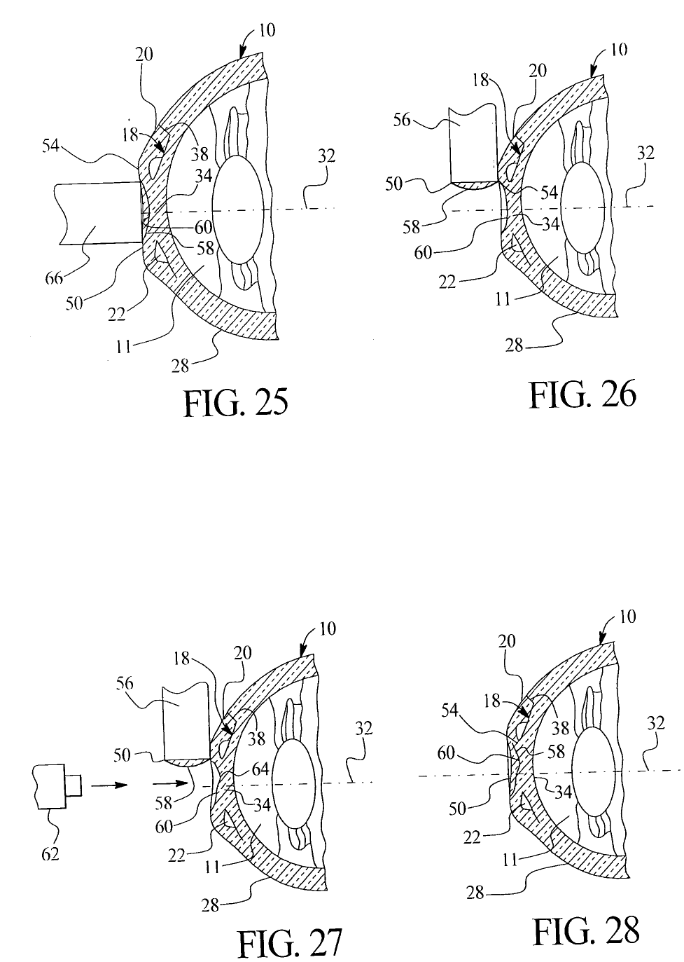

### Embodiment of FIGS. 25-28

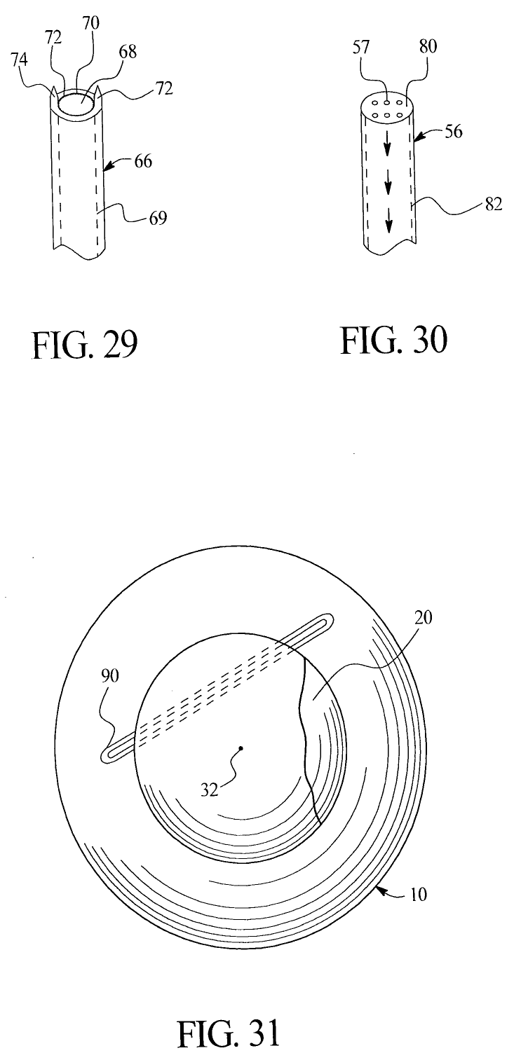

In a further embodiment, a second flap 50 can be formed from the corneal epithelium on the surface 52 of the cornea 20, a seen in FIGS. 25-28 to reduce or eliminate irregularities in the healing of the cornea. Preferably, the flap is formed overlying portion 34 using a device 66 that has a sponge 68 thereon. As seen in FIG. 29, device 66 is a cylindrical tube having an opening 70 with sponge 68 inserted therein. Alcohol is fed through a hollow portion 69 that runs longitudinally along the interior of device 66. When the alcohol saturates the sponge, the sponge can be applied to the surface of the cornea. The alcohol loosens the epithelium from the basement membrane, which allows removal of the epithelial layer. If it is desired to have the flap a least partially attached as shown in FIGS. 25-28, by portion 54, a notch 72 can be formed along the edge of device 66, thereby preventing the sponge from contacting potion 54. Furthermore, the device 66 can have spikes or markers 74 at predetermined points on the edge of the device. For example, the markers can be at 90 degree or 180 degree intervals. The spikes can have a stain, such as gentian violet or any other suitable dye, applied thereto, so that the exact location and orientation of the flap is known. Therefore, when the flap is replaced or reapplied, it can be replaced in the exact or at least the substantially same position from which it was removed.

The second flap 50 is a relatively small flap that preferably at least partially overlies or is concentric about the visual axis or main optical axis 32 and can be attached to the cornea 20 by portion 54. However, the flap can be formed on any portion of the cornea desired and in any suitable manner, such as with a knife or laser. It is noted, that the location of the flap does not necessarily need to be concentric about the main optical axis and can be at any location on the surface of the eye and may be any size desired.

The flap is preferably pealed or moved away from the surface of the cornea using a suction device 56 (FIG. 30), but may be removed using any other device known in the art. As seen in FIG. 30, device 56 is substantially cylindrical with air holes 57 extending through top surface 80. When suction device 56 is used, the flap is moved away from the cornea and remains attached to device 56. Generally suction is applied and air travels through passageway 82, which extends longitudinally along the interior of device 56. When surface 80 is applied to the portion of the epithelium that has had alcohol applied thereto, a vacuum is formed in passageway 82 and flap 50 can be removed, as seen in FIGS. 26 and 27, exposing third and fourth internal corneal surfaces 58 and 60. Surface 58 generally faces in a posterior direction and surface 60 generally faces in an anterior direction.

Once the flap is moved to expose surfaces 58 and 60, an excimer laser 62, as seen in FIG. 27, can be used to ablate a portion 64 of the cornea 20 to reduce or eliminate any remaining refractive error. Portion 64 is preferably a portion of the Bowman's layer or basement membrane, but can be any portion of the cornea desired. The flap 50 is then replaced and allowed to heal as seen in FIG. 28. The flap may simply be placed over the ablated portion and heal or it may be affixed thereto in any manner known in the art, such as by sutures or adhesive.

When performing the excimer laser procedures described above and shown in FIGS. 23 and 27, it is possible to simultaneously use wavefront technology or Adaptec optic technology to create a near perfect correction in the eye and to remove all corneal irregularities. By using this technique to correct vision, it is possible to achieve 20/10 vision in the patient's eye or better.

The patient can undergo the second laser ablation, as seen in FIGS. 23 or FIG. 27, either immediately after the insertion of the ocular implant or after a substantial time difference, such as days or weeks later, and any step or portion of the above procedure may be repeated to decrease the refractive error in the eye.

After the above procedures are preformed, and the ocular material is in place, if necessary, a flap 42 can be formed in the surface of the cornea of the eye, which would expose the ocular material 22 when removed or folded away, as seen in FIG. 24. Once the flap is removed or folded away, the ocular material can be irradiated and a portion 44 or the material 22 ablated by an excimer laser 46 and wavefront technology, as described above. Preferably, this technique is used on the pocket having no portion attached in the center, but may be used with any type of pocket, including the ring-shaped pocket.

Furthermore, at the end of the procedure or before the ablation of the surface of the cornea, topical agents, such as an anti-inflammatory, antibiotics and/or an antiprolifrative agent, such as mitomycin or thiotepa, at very low concentrations can be used over the ablated area to prevent subsequent haze formation. The mitomycin concentration is preferably about 0.005-0.05% and more preferably about 0.02%. A short-term bandage contact lens may also be used to protect the cornea. The short term contact lens specifically protects the portion of the cornea that has flap 50 formed thereon, but also can protect the cornea after any of the above steps in this procedure.

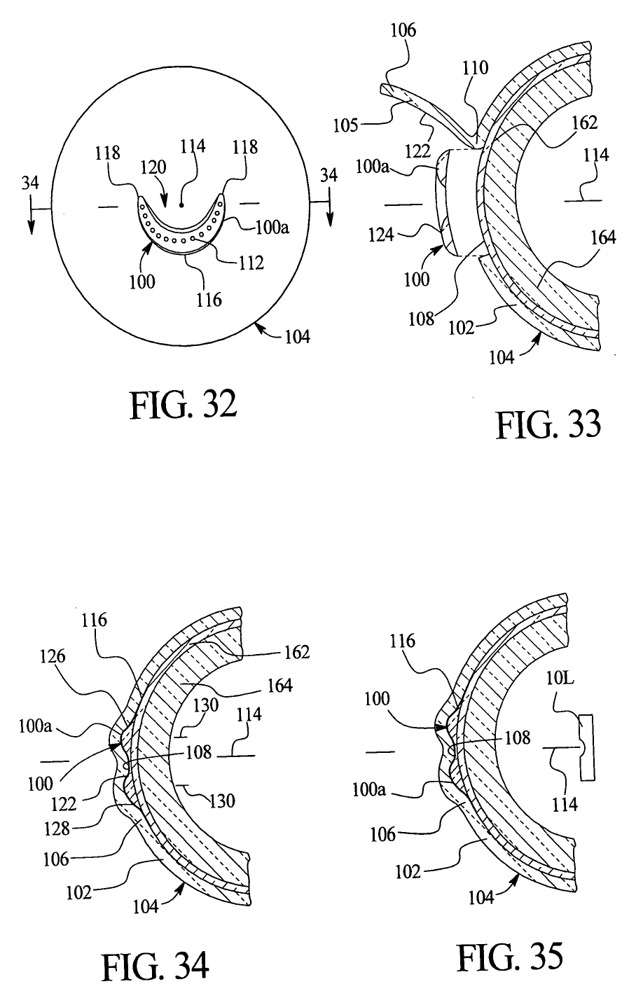

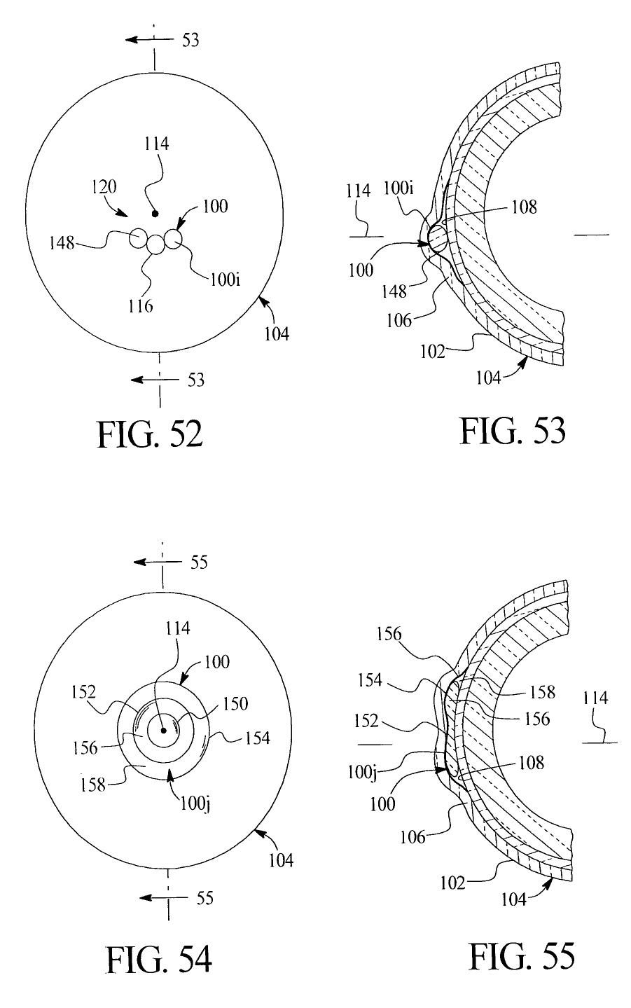

### Embodiments of FIGS. 32-64

Referring to FIGS. 32-62, treatment of a refractive error such as presbyopia, is also accomplished by implanting a biocompatible inlay 100, such as a lens or other ocular material, under the epithelium 102 of the cornea 104. In general, a surface 105 of epithelium 102, such as a flap, is separated from a corneal surface 108 of cornea 104 so that inlay 100 can be implanted between the surface of the epithelium 102 and the corneal surface 108. This treatment method maintains the integrity of the cornea 104 by only cutting into the epithelium 102, allowing the inlay 100 to be easily implanted and removed, and reducing scarring. Also, by not discarding the portion of the epithelium that has been removed, irregularities in the healing of the cornea that often occur during regrowth of the epithelium 102 are minimized. Although this treatment method is preferably used to correct presbyopia, the method can be employed to correct any refractive error including hyperopia, myopia and astigmatism.

Preferably, the surface 105 of epithelium 102 that is separated from corneal surface 108 forms an epithelial flap 106. Inlay 100 can then be implanted on a corneal surface 108 exposed by the separation of epithelial flap 106. Epithelial flap 106 is replaced in tact over inlay 100 and corneal surface 108 with epithelial flap 106 conforming to the shape and curvature of inlay 100. Epithelial flap 106 preferably remains attached to epithelium 102 at a peripheral area of the flap, forming hinge 110, as seen in FIG. 33. However, flap 106 can be completely detached from epithelium 102 and then replaced over inlay 100 and corneal surface 108 or flap 106 can be attached at any portion of the cornea desired, such as at the main optical axis. Alternatively, instead of a flap 106, a pocket can be formed between epithelium 102 and corneal surface 108 that receives inlay 100 at an opening of the pocket. Although it is preferable to form the flap 106, or pocket, in the epithelium 102, the flap 106 can be formed in other layers of cornea 104 including the Bowman's layer 162 or the stroma 164, such as is done in the LASIK procedure.

FIGS. 32-55 illustrate various examples of inlay 100, including inlays 100a-100j, respectively. Each inlay has a particular shape and curvature to provide correction for a particular type of refractive error. Generally, inlays 100a-100j, are small with a diameter ranging between 1-7 mm, and preferably 3-4 mm. Diffractive technology allows the inlays to be made very thin with a thickness ranging between 0.1-200 microns. The thin nature of inlays 100a-100j facilitates implantation of each inlay 100 under epithelium 102. Micro perforations 112 can be included in inlays 100, as seen in FIG. 32, for example (illustrating inlay 100a), to facilitate fluid or nutrient flow through the inlays, which reduces or eliminates cloudiness or opacification or potential necrosis caused by malnutrition. To correct a variety of refractive errors, inlays 100a-100j can have plus, minus or astigmatic power or any combination thereof such as to create a bifocal effect.

Also, inlays 100a-100j are similar to ocular material 22 (see FIGS. 9-18) and likewise can have a variety of shapes, a uniform or varied thickness, single or multiple layers, or multiple sections that are either integral or separate, and can be either concentric or eccentric with the visual axis 114. The shaped and curvature of each inlay is predetermined based on the refractive error or errors that require correction.

Each inlay 100a-100j preferably includes a blend zone or area 116 which eliminates square edges to provide a gradual change or slope between each inlay and corneal surface 108, thereby reducing discomfort to the patient. Generally, blend zone 116 surrounds the peripheral edges of each inlay, as seen in FIG. 32, for example (illustrating inlay 100a).

As seen in FIGS. 32-37, inlay 100a is substantially semi-circular or semi-ring shaped. Ends 118 of inlay 100a are preferably tapered, as seen in FIG. 32. Inlay 100a is preferably concentric with visual axis 114 of cornea 104 and leaves uncovered an annular area 120 surrounding visual axis 114. Optionally, micro perforations 112 are disposed along inlay 100a.

Once inlay 100a is implanted on corneal surface 108, as seen in FIG. 33, flap 106 is replaced over implant 100a and corneal surface 108, as seen in FIG. 34, so that posterior surface 122 of flap 106 directly overlies the front surface 124 of inlay 100a. Flap 106 conforms to the shape of inlay 100a. The difference in shape, including the difference in curvature, between inlay 100a and cornea 104 provides either plus power for correcting farsightedness, minus power for correcting nearsightedness, or an astigmatic power for correcting an astigmatism. For example, as seen in FIG. 34, in cross-section, inlay 100a includes first and second curved portions 126 and 128 which each define a curvature about their respective central axis 130 that is substantially greater than the curvature of cornea 104, as defined about visual axis 114. This difference in curvature provides plus and minus correction of the refractive error.

Preferably, the refractive index of inlay 100a is the same as the refractive index of cornea 104, thereby relying on the difference in curvature and shape between inlay 100a and cornea 104 to provide the appropriate refractive correction. However, inlay 100a can have a different index of refraction than cornea 104. Also, as seen in FIG. 37, inlay 100a can include multiple layers 132 and 134 each having either the same or different index of refraction from cornea 104.

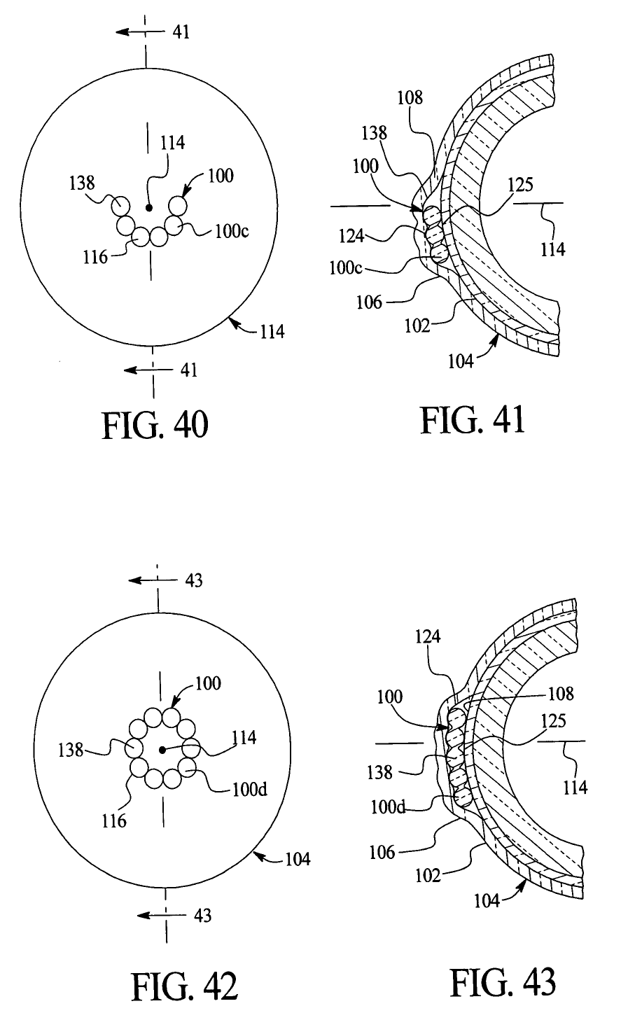

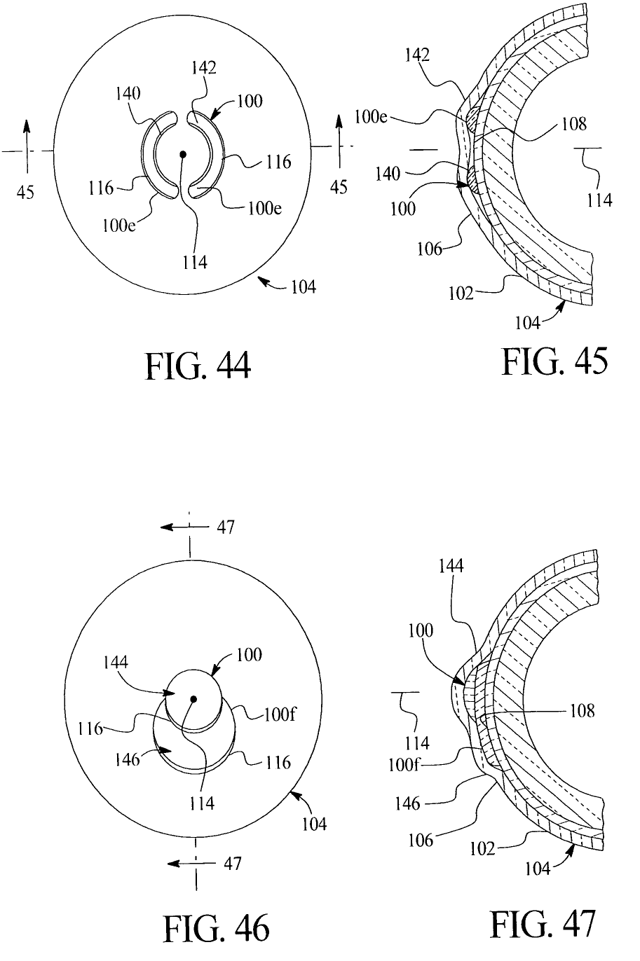

As seen in FIGS. 38-45, inlays 100b-100e each provide correction of refractive errors based on their curvature and shape, similar to inlay 100a. Each inlay 100b-100e is preferably concentrically disposed with respect to visual axis 114 on corneal surface 108. Inlay 100b is substantially ring-shaped (see FIG. 38) and includes curved portions 136 in cross section similar to curved portions 126 and 128 of inlay 100a (see FIG. 39). Inlays 100c and 100d are formed of a plurality of segments 138 each generally circular in shape, with inlay 100c having a semi-ring shape (see FIG. 40) and inlay 100d having a ring shape (see FIG. 42). In cross section, segments 138 define a plurality undulations on both the front and back surfaces 124 and 125 of inlays 100c and 100d (see FIGS. 41 and 43). Inlay 100e includes two separate sections 140 and 142 (see FIG. 44) each similar in curvature and shape to inlay 100a (see FIG. 45). As seen in FIGS. 39, 41, 43 and 45, epithelial flap 106 conforms to the shape and curvature of each inlay 100b-100e including curved portions 136 of inlay 100b, the undulations of surfaces 124 and 125 of inlays 100c and 100d, and the curved sections 140 and 142 of inlay 100e.

As seen FIGS. 46-47, inlay 100f also provides correction of refractive errors in the same manner as described above with respect to inlays 100a 100e, and also preferably includes multiple sections 144 and 146 integrally attached to form inlay 100f. Specifically, the first and second sections 144 and 146 are generally circular in shape with first section 144 having a smaller diameter than second section 146 (see FIG. 46). Only first section 144 is concentric with visual axis 114, as seen in FIG. 47, however, either section can be concentric or eccentric with respect to axis 114. First section 144 overlies second section 146 with each section preferably providing correction for a different refractive error by the curvature and shape of each section 144 and 146. For example, first section 144 corrects myopia or hyperopia and second section 146 corrects for presbyopia. However, the shape and curvature of each section 144 and 146 can be changed to provide correction for any refractive error. Epithelial flap 106 conforms to the shape of each section 144 and 146 including the more convex curvature of section 144 as compared to section 146.

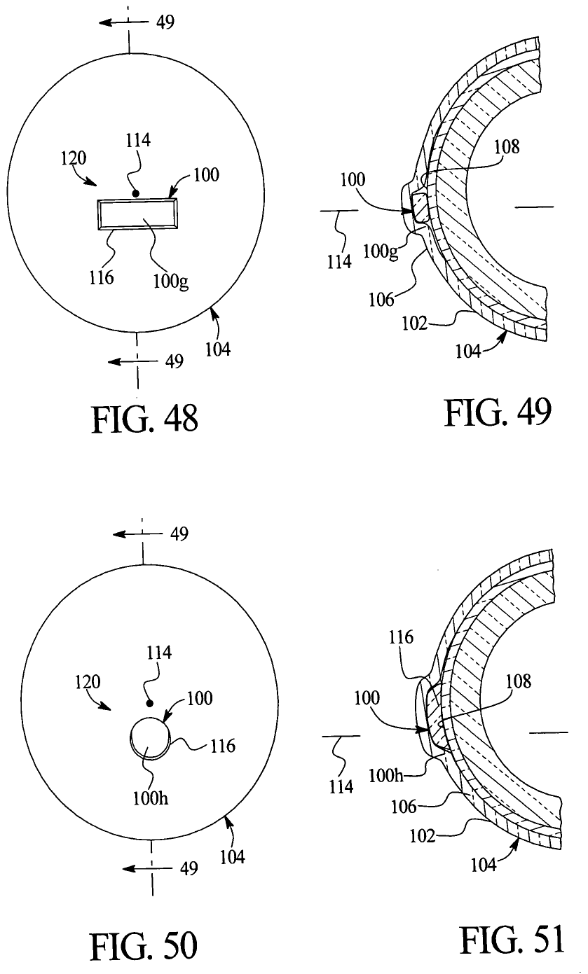

As seen in FIGS. 48-53, inlays 100g-100i provide correction of refractive errors in the same manner as described for inlays 100a-100f and are preferably eccentric to visual axis 114 but located in annular area 120 defined around axis 114 (see FIGS. 49, 51 and 53). Inlay 100g is non-circular and generally rectangular in shape, as seen in FIG. 48. However, inlay 100g can be any polygonal non-circular shape. Epithelial flap 106 conforms to the shape of inlay 100g including the substantially square cross sectional shape and blend zone 116 of inlay 100g, as seen in FIG. 49. Inlay 100h is generally disc shaped, as seen in FIG. 50. As seen in FIG. 51, epithelial flap 106 conforms to the shape of inlay 100h including the flat curvature and blend zone 116 of inlay 100h. Inlay 100i is formed of a row of segments 148 similar to segments 138 of inlays 100c and 100d. Epithelial flap 106 conforms to the shaped of each segment, as seen in FIG. 53.

As seen in FIGS. 54-55, inlay 100j is formed of first, second and third circular sections 150, 152 and 154. First section 150 is generally disc shaped with second section 152 surrounding first section 150 to form a first ring 156 of inlay 100j and third section 154 surrounding second section 152 to form a second ring 158. First, second and third sections 150, 152 and 154 preferably provide correction for different refractive errors in an alternating manner. For example, first section 150 has a flatter curvature than second section 152 or first ring 156, as seen in FIG. 55, to provide correction for myopia, second section 152 has a more convex curvature to provide correction for presbyopia and hyperopia, and third section 154 or second ring 158 has a flatter curvature to provide correction for myopia. Additional sections or rings can be added to inlay 100j to continue providing refractive error correction in an alternating manner. As seen in FIG. 55, epithelial flap 106 conforms to the variations in curvature of each section 150, 152 and 154, i.e. flatter versus more convex curvature.

When treating presbyopia, the inlay 100, such as inlay 100a-100j, preferably does not cover annular area 120 around visual axis 114 of cornea 104, such as seen in FIGS. 32, 38, 42 and 44, for example. This uncovered area 120 allows the patient to see through the uncovered annular area 120 for normal far vision and see through the inlay for correction of presbyopia when the patient reads. As a result of the uncovered annular area 120, the presbyopia of the patient is corrected without distorting the patient's normal far vision.

Correction of presbyopia and/or hyperopia is provided in two ways. The first way is by the index of refraction of the inlay 100, as described above. Specifically, by using an inlay 100, such as one of inlays 100a-100j, that has a higher index of refraction than the cornea 104, plus correction for presbyopia is provided. The difference in the index of refractions between the inlay and the cornea corrects the refractive error due to the inlay 100 bending light differently, i.e. refracts closer to the cornea, than the cornea. Examples of materials used to form the inlay that have an index of refraction different or higher than the cornea include silicone, methacrylate, hydrogel, hilafilcon, or mixture of various synthetic and/or organic polymers.

The second way of correcting presbyopia and/or hyperopia is to provide inlay 100 with a curvature that is different than the curvature of cornea 104. Preferably, inlay 100 has at least a portion with radial curvature that is smaller than the radial curvature of cornea 104, thereby correcting presbyopia and/or hyperopia by bending more light closer to the cornea. The smaller the radial curvature of inlay 100 with respect to cornea 104 the more correction is provided for presbyopia and/or hyperopia, as is well known in the art.

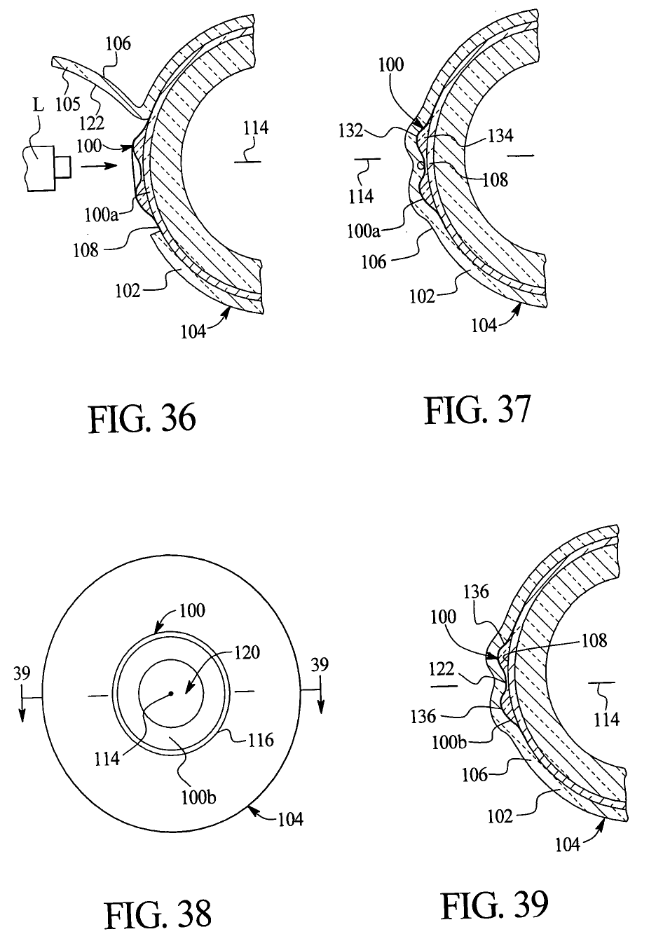

As described in U.S. Pat. No. 6,436,092 to Peyman, which is herein incorporated by reference, a laser L can alternatively be employed to ablate inlays 100a-100j or the surrounding area of the cornea prior to replacing epithelial flap 106, as seen in FIG. 36 for example, or inlays 100a-100j can be adjusted after epithelial flap 106 is replaced using light, such as infrared light, when additional correction of a refractive error is required, as described in U.S. patent application Ser. No. 09/494,248, which is herein incorporated by reference. Also, an intraocular lens IOL can be used in combination with inlays 100a-100j, as seen in FIG. 35, to create a telescopic effect.

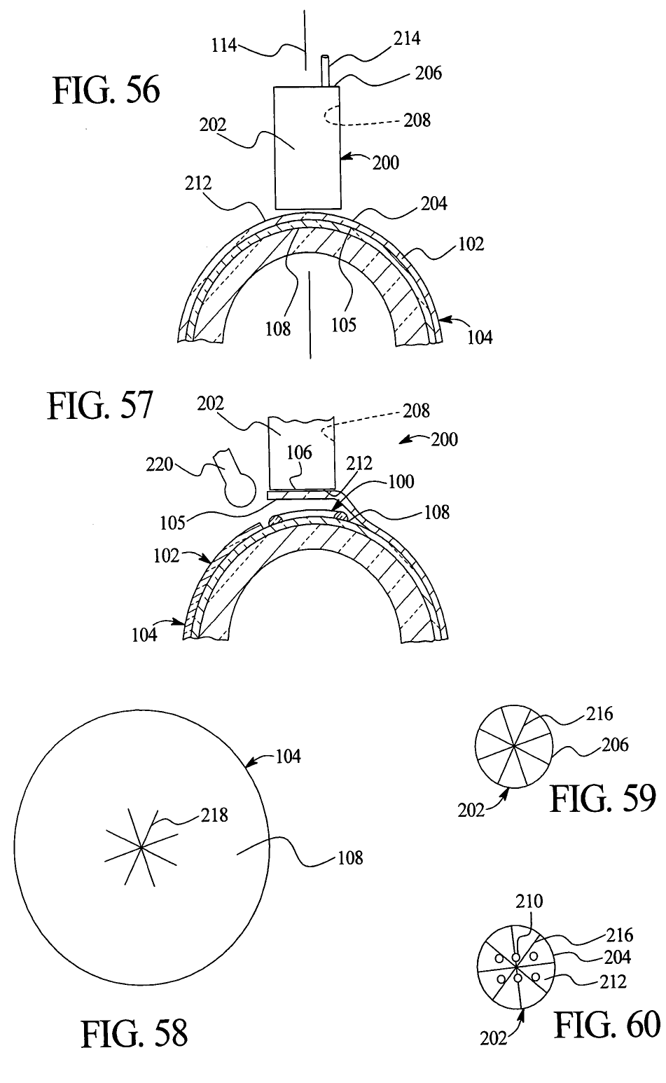

Referring to FIGS. 56-60, epithelial flap 106 is formed by applying suction to a portion of the epithelium 102 using a suction device 200 to separate the epithelium surface 105 from corneal surface 108. As seen in FIGS. 56-60, suction device 200 is generally a cylindrical chamber 202 having first and second opposing walls 204 and 206 and defining an internal area 208. Suction device 200 operates in generally the same manner as suction device 66, as seen in FIGS. 31. First wall 204 of suction device 200 includes aeration holes 210 (see FIG. 59) and an engagement surface 212 that contacts and supports the epithelial flap 106 once separated from corneal surface 108, as seen in FIG. 57. At second wall 204, suction 214 is applied through internal area 208 by a suctioning mechanism such as a vacuum. Additionally, markers or spikes 216 similar to markers 74 described above with respect to suction device 66, are included at first wall 204 to mark the exact location and orientation of flap 106, particularly with respect to visual axis 114. Chamber 202 is preferably transparent allowing visual observation of markers 216 as well as the corresponding marks 218 left on epithelial flap and cornea 108, as seen in FIG. 58.

Once suction 214 is applied to epithelium 102 at the desired location of flap 106, i.e., preferably centered with respect to visual axis 114, a cutting device 220 is employed to dissect flap 106 from corneal surface 108. The operator can observe as cutting device 220 through transparent chamber 202 as device 220 is dissecting flap 106. Cutting device 220 is preferably a spatula but can be any cutting device known in the art, such as a knife or microkeratome. Due to the suction 214 applied through internal area 208 and aeration holes 210 of chamber 202, flap 106 will lift and separate from corneal surface 108 and abut engagement surface 212. Alcohol can also be used to facilitate this process. Flap 106 remains engaged with surface 212 of chamber 202 keeping the flap 106 in tact. Once the inlay 100, which is preselected, such as from inlays 100a-100j, is implanted on corneal surface 108, flap 106 can be replaced over the inlay 100 and corneal surface 108. By looking through transparent chamber 202, markers 216 can be matched with marks 218 on the corneal surface to ensure proper positioning of flap 106. This procedure maintains the integrity of the epithelium and avoids the need to regrow the epithelium over the implanted inlay and thus avoids irregularities that often result therefrom.

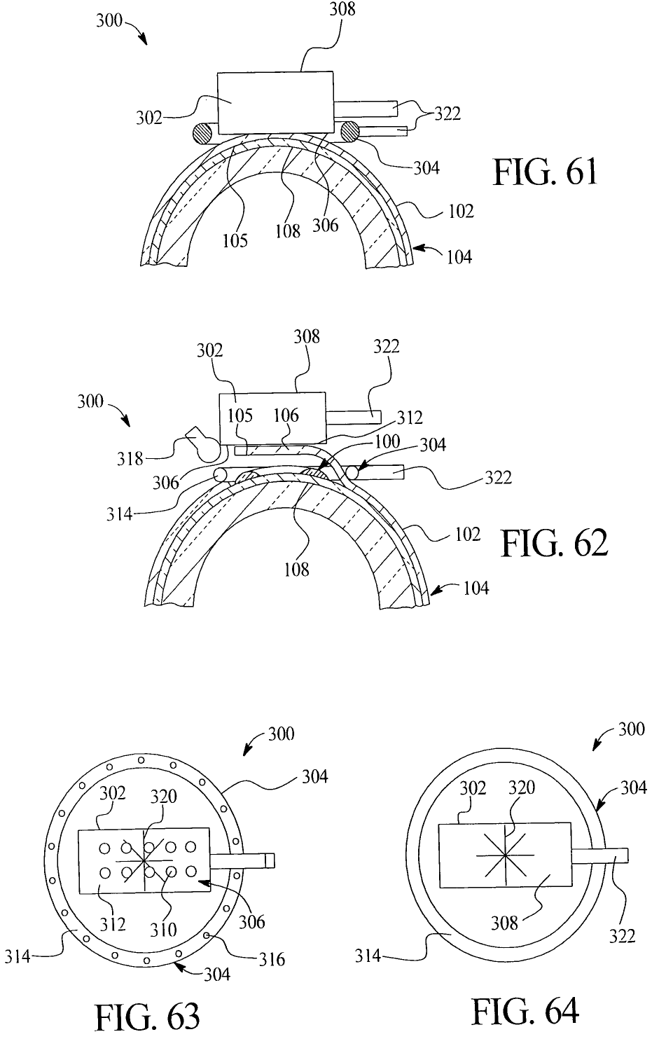

Referring to FIGS. 61-64, an alternative suction device 300 is disclosed that employs both a chamber 302 for supporting the epithelial flap 106 and a holding device 304 for holding a portion of the eye surrounding the flap 106. Chamber 302 is similar to chamber 202 except that chamber 302 is generally rectangular in shape. Chamber 302 is preferably transparent including opposite first and second walls 306 and 308 with the first wall 306 including aeration holes 310 and an engagement surface 312. Holding device 304 is preferably a tubular ring 314 having aeration holes 316 to engage and hold the eye around flap 106 while dissecting flap 106. An automatic cutting device 318 is preferably used, such as a vibrating spatula, that dissects the epithelial flap inside of tubular ring 314 and under chamber 302. Epithelial flap 106 will lift and separate from corneal surface 108 and engage engagement surface 312, as seen in FIG. 62, in the same manner as described above with respect to device 200 using suction 322. Also, as with device 200, the transparent nature of the chamber 302 allows the operator to observe cutting device 318 as flap 106 is being dissected and match markings 320 of chamber 302 with marks of the corneal surface.

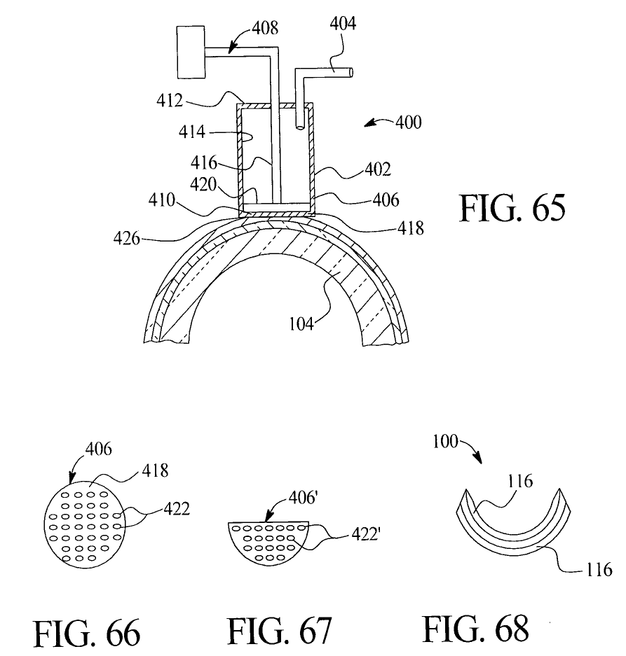

Referring to FIGS. 65-67, another alternative device 400 for creating a flap in cornea 104 in accordance with the present invention generally includes a chamber 402, a suction device 404, a plate 406 and a vibrating device 408. Chamber 402 includes first and second opposite ends 410 and 412 and an interior area 414 that supports plate 406 at first end 410. Vibrating device 408 is coupled to plate 406 and vibrates plate 406 within chamber interior area 414. Specifically, vibrating device 408 includes an arm 416 that extends through chamber second end 412, into interior area 414 and attaches to plate 406 in any conventional manner. Vibrating device 408 is preferably any conventional vibrating mechanism known in the art and/or other arts, such as vibrating toothbrushes and shavers.

Chamber interior area 414 supports plate 406 by an inner flange 416 of chamber 402 extending into interior area 414 at chamber first end 410 so that plate 406 can freely vibrate via vibrating device 408. However, any conventional coupling mechanism can be employed to support plate 406 within interior area 414, as long as plate 406 is allowed to vibrate. First end 410 of chamber 402 is open thereby exposing a cornea engagement surface 418 outside of interior area 414 for engaging corneal surface 108. Opposite cornea engagement surface 418 is vibrating device attachment surface 420 of plate 406 for engaging arm 416 of vibrating device 408 as described above.

As seen in FIG. 66, plate 406 is substantially circular and forms a flap 426 in generally the same manner as described above having a circular shape corresponding to the shape of plate 406. Plate 406 includes a plurality of aeration holes 422 in fluid communication with a suction 424 of suction device 404 that extends through chamber second end 412 and into interior area 414. Although plate 406 is preferably circular in shape, plate 406 can have any desired shape. For example, plate 406′ shows an alternative shape for plate 406 as substantially semi-circular with aeration holes 422′.

Flap 426 is similar to flap 106 described above and is formed using device 400 by placing chamber first end 410 and plate 406 on the epithelium 102 of cornea 104. Application of suction to chamber interior area 414 via suction device 404 draws flap 426 into engagement with cornea engagement surface 418 of plate 406 via aeration holes 422. Vibrating plate 406 via vibrating device 418 separates flap 426 from cornea 104 allowing implantation of an inlay, as described above. Alternatively, a vibrating spatula or a knife can be employed to separate flap 426 once plate 406 engages flap 426. Although it is preferable to create a flap 426, device 400 can also be used to create a pocket (not shown) in cornea 104. Also, flap 426 is preferably formed by separating the epithelium 102 from cornea 104, however, flap 426 can be created in any layer of the cornea 104.

As seen in FIG. 68, blend zone 116 of inlays 100a-100j is painted with a light absorbing pigment (FIG. 68 showing only inlay 100a). The pigmented blend zone 116 prevents glare that is often a result of implantation of the inlay, such as inlays 100a-100j, being implanted in the cornea.

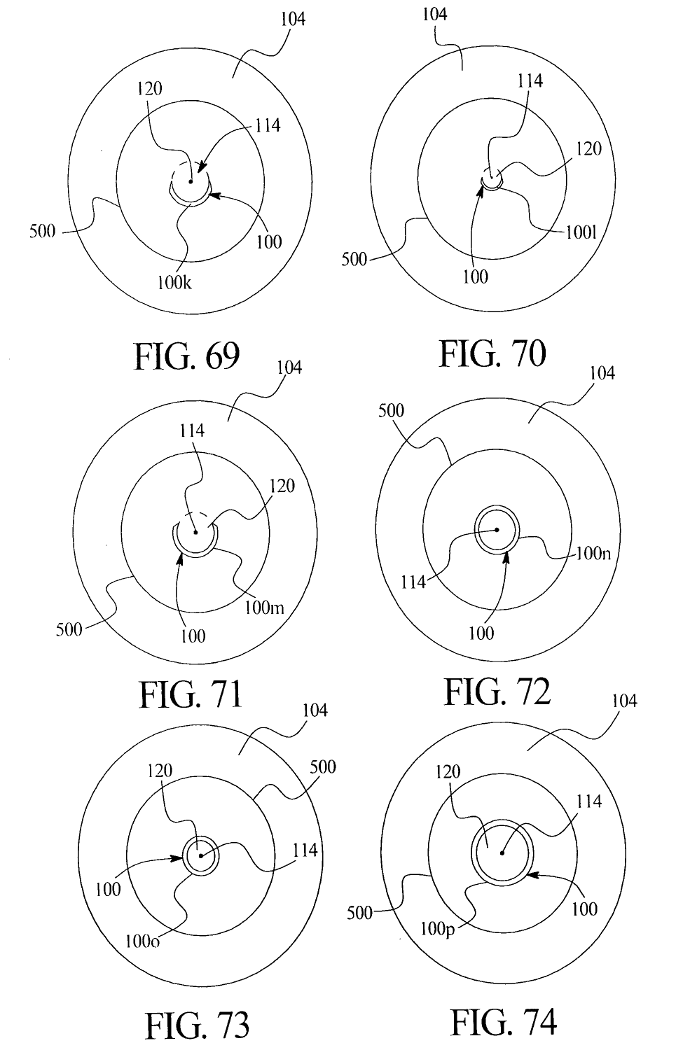

To evaluate and select the most appropriate inlay 100 for a particular patient, a lens 500, such as a contact lens, is preferably used that supports the inlay 100 selected from a group of inlays 100 to be tested on the patient. The groups of inlays include, for example, inlays 100a-100j, as seen in FIGS. 32, 38, 40, 42, 44, 46, 48, 50, 52, 54, and inlays 100k-100u shown in FIGS. 69-78. A variety of inlays 100 which have different shapes and sizes, and which can also vary in distance from the central visual axis 114 of the cornea 104 can be used with contact lens 500 to test for refractive errors and determine the appropriate inlay 100 for a particular patient. Inlays 100a-100u are examples of inlays 100 that can be used with contact lens 500. Once an inlay 100 is selected from the group of inlays, the inlay is coupled to contact lens 500 and placed on the patient's cornea 104 to determine whether that selected inlay is appropriate for correcting the refractive error of the patient. Specifically, contact lens 500 with inlay 100 coupled thereto is oriented over the central visual axis 114, so that inlay is disposed adjacent annular area 120 surrounding visual axis 114 and so that only contact lens 500 covers annular area 120. As described above, the patient is able to see through annular area 120 for normal far vision and see through inlay 100 for reading and correction of the patient's presbyopia. Either a single contact lens 500 supporting a selected inlay 100 one at a time or multiple contact lenses 500 each supporting a different selected inlay 100 can be used to evaluate the appropriate inlay 100 for the patient.

As seen in FIGS. 69-78, each inlay 100k-100u is supported by a contact lens 500 and placed on the patient's cornea 104 for testing. As seen in FIGS. 69-71, each of inlays 100k-100o is substantially semi-ring shaped, similar to inlay 100a. Each inlay 100k-100o is generally concentric with visual axis 114 and located adjacent annular area 120. Inlay 1001 has a smaller radial curvature that inlay 100k and is disposed closer to visual axis 114 than inlay 100k. Conversely, inlay 100o has a larger radial curvature than inlay 100k and is disposed further away from axis 114.

As seen in FIGS. 72-74, each of inlays 100m-100p is substantially ring-shaped, similar to inlay 10b. Each inlay 100m-100p is generally concentric with visual axis 114 and extends around annular area 120 leaving annular area 120 uncovered by the inlay. Inlay 100n has a smaller radius than inlay 100m and is thus closer to visual axis 114 than inlay 100m. Inlay 100p has a larger radius than inlay 100m and is further away from visual axis 114 than inlay 100m.

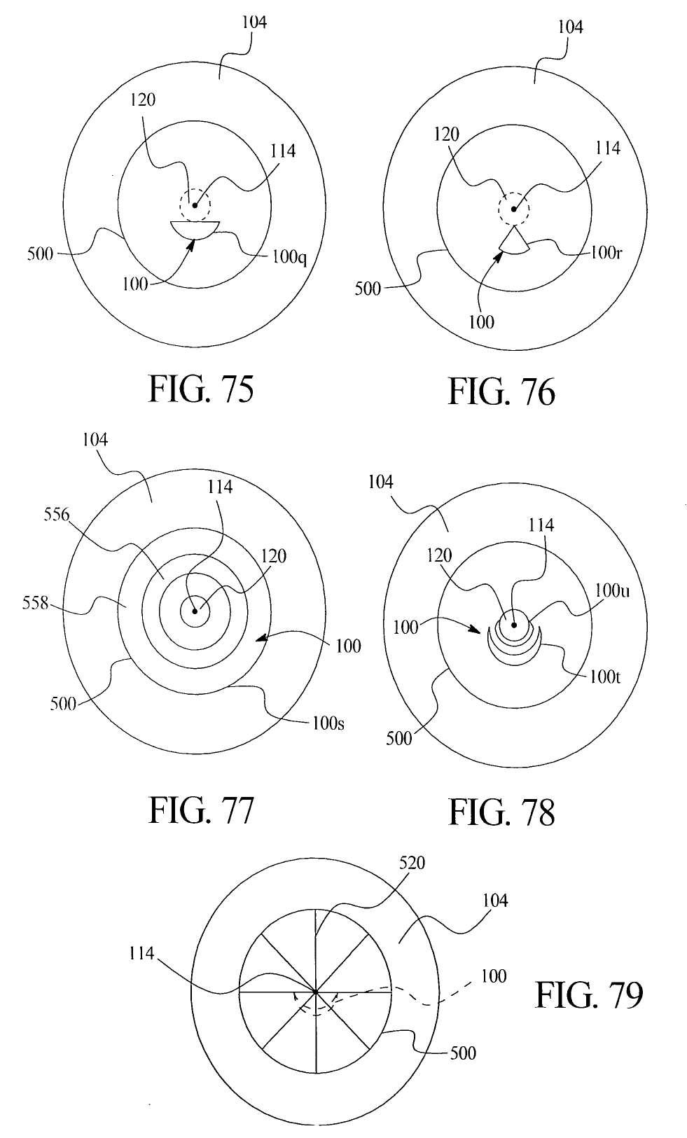

As seen in FIG. 75, inlay 100q is substantially semi-circular shaped and is oriented adjacent annular area 120. Inlay 100r is substantially triangular in shape and disposed adjacent visual axis 114, as seen in FIG. 76. Inlay 100s is similar to inlay 100j, and includes multiple rings 556 and 558 concentric with visual axis 114 and surrounding annular area 120. As seen in FIG. 78, two inlays 100t and 100u are combined on contact lens 500. Inlay 100u is semi-ring shaped and has a smaller radial curvature than inlay 100t which is also semi-ring shaped. Thus inlay 100u is disposed adjacent annular area 120 and closer to visual axis 114 with inlay 100t being spaced from inlay 100u.

Contact lens 500 is made of a flexible compatible material that is synthetic, organic or a combination thereof. Contact lens 500 is marked, as seen in FIG. 79, to correspond to die markings of the cornea. This allows contact lens 500 and the selected inlay to be precisely centered on cornea 104 with respect to visual axis 114.

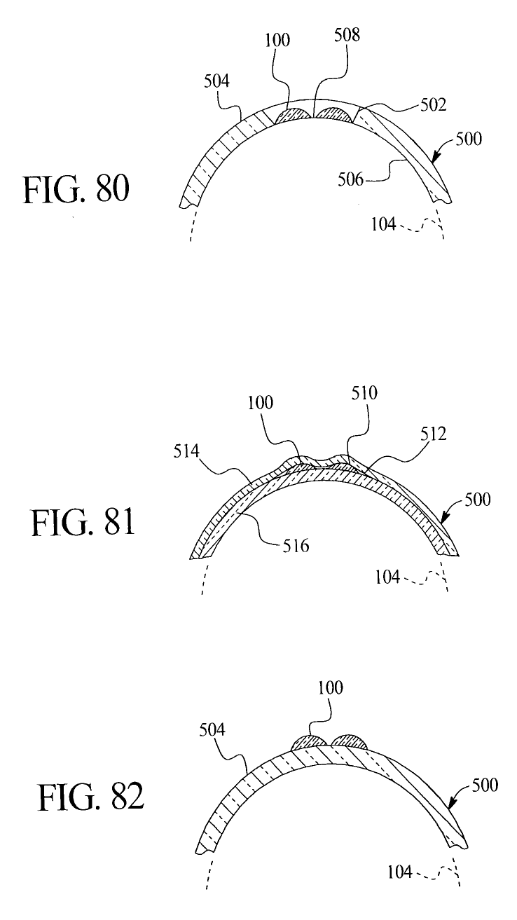

Inlay 100 is coupled to contact lens 500 in one of three ways. In the first way, inlay 100 is placed within a recess or window 502 of contact lens 500, as seen in FIG. 80. Recess 502 is open at an outer surface 504 of lens 500 opposite an inner surface 506 for engaging the cornea 104. Recess 502 extends into contact lens 500 and includes an inlay supporting surface 508 upon which the selected inlay 100 rests. If the selected inlay 100 is not appropriate for the patient, that inlay can be removed from recess 502 and another selected inlay can be placed in the recess 502. Thus recess 502 allows multiple inlays to be individually received therein and tested on the patient, and removed without having to remove lens 500 from the patient's cornea, thereby allowing testing of various inlays 100 until the appropriate one for the patient is found.

The second way to couple the selected inlay 100 with contact lens 500 is to implant inlay 100 between first and second surfaces 510 and 512 of lens 500. Preferably, first and second surfaces 510 and 512 are disposed on first and second layers 514 and 516, as seen in FIG. 81, so that inlay 100 is embedded inbetween the layers 514 and 516. The third way to couple the selected inlay 100 with lens 500 is to attaching inlay 100 to outer surface 504 using a bioadhesive.

A patient with presbyopia is examined, and corrected for far vision if required and the degree of presbyopia is determined. An inlay 100 is selected from the group of inlays and coupled to lens 500, as described above. Lens 500 with the selected inlay 100 is centered on the patient's cornea 104 to determine whether that selected inlay is appropriate for the patient. This process is repeated with different inlays coupled to lens 500 until the appropriate inlay is found for the patient. The patient will choose the best add or inlay embedded in the contact lens 500 that the patient prefers and that provides the near vision or correction for presbyopia without producing too much of glare or blurring of the far vision. For example, depending on the amount of correction required, some patients may prefer an inlay 100 that is closer to visual axis 114, such as inlays 1001 or 100o, thereby providing more correction for presbyopia. If correction for far vision is not required, the contact lenses 500 tested on the patient would be those that would not cover the annular region around the visual axis 114. Then the selected contact lens is positioned on the central visual axis on the patient's cornea. Then the position of the add or lens on the cornea is marked with the dye markings 520 with respect to visual axis 114 (see FIG. 79) for subsequent implantation of the selected inlay. The selected inlay 100 will be implanted in the manner described above in the same orientation as the tested contact lens using the markings, such as at the same distance from the visual axis 114.

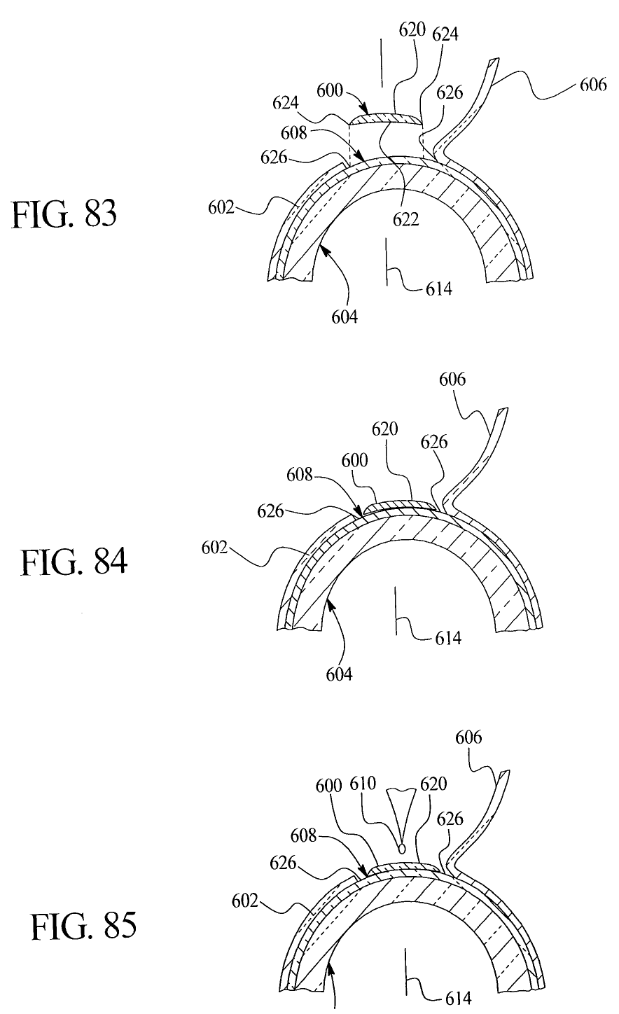

### Embodiment of FIGS. 83-88

Referring to FIGS. 83-88, an inlay 600 is implanted in a patient's cornea 604 to correct refractive errors in the same manner as described above with respect to inlay 100 and cornea 104 of the embodiments of FIGS. 32-64, except an immobilizing coating 610 is applied to inlay 600 to ensure that the proper position of inlay 600 with respect to the visual axis is maintained. Coating 610 can be applied to inlay 600 during the implantation process. As with the embodiments of FIGS. 32-64, cornea flap 606 is preserved, instead of removing flap 606 and waiting for flap 606 to grow back over inlay 600. Inlay 600 can be implanted in any layer of the cornea including the epithelium or stroma.

Inlay 600 may be any shape such as disc (FIG. 83), ring (FIG. 38), or semi-ring (FIG. 32) shaped, or any of the shapes of inlays 100a-100j. Inlay 600 is formed in the same manner as inlay 100 to correct refractive errors, such as myopia, hyperopia, and presbyopia. Also, as with inlay 100, inlay 600 can be made very thin, such as 0.1-200 microns, using diffractive technology. Inlay 600 can include pores like pores 112 of inlay 100 (FIG. 32) to facilitate the flow of nutrients between the corneal layers of cornea 604 and through inlay 600. The pores can have a size of 0.6-50 micrometers, and preferably 1-2 micrometers. Inlay 600 may be formed of organic materials, such as collagen, laminin, or vitronectin, or synthetic materials, such as silicon, hydrogel or hilaficon, or a mixture of organic and synthetic materials. Rather than single focality, inlay 600 can be formed with the characteristics of a diffractive beam splitter to generate multifocality, i.e., far, middle, and near vision.

As seen in FIG. 83, inlay 600 has a front surface 620, an opposite back surface 622, and side surfaces 624 extending therebetween. Flap 606 can be formed in a layer 602 of cornea 604 in the same manner as described for the embodiments of FIGS. 1-82. Layer 602 may be the epithelium of cornea 604 or any other layer, such as the stroma, of cornea 604. Flap 606 is lifted and pulled back, thereby exposing corneal surface 608. Inlay 600 is then implanted in cornea 604 by placing inlay 600 on exposed corneal surface 608 with back surface 622 resting on corneal surface 608, as seen in FIG. 84.

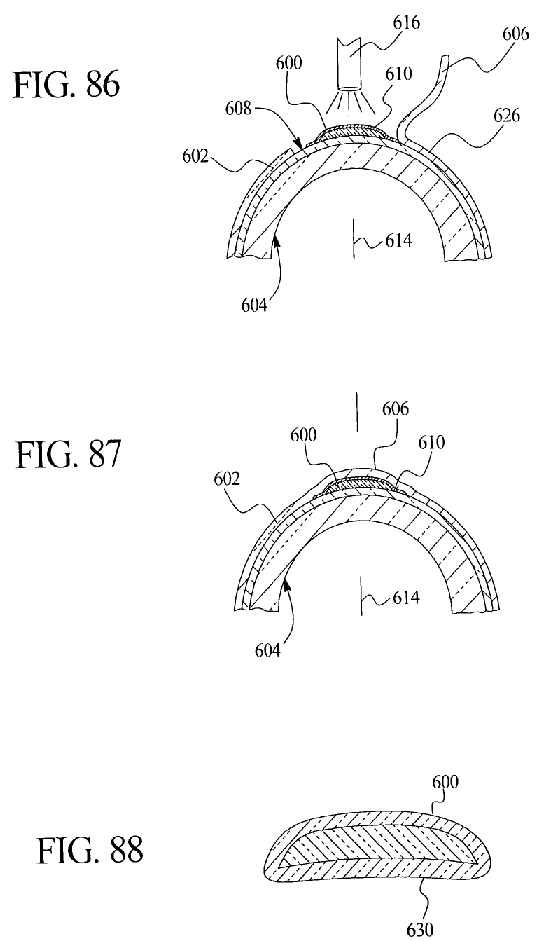

Inlay 600 is then positioned or centered with respect to visual axis 614 in the same manner as described above with respect to the embodiments of FIGS. 32-64. Inlay 600 can be ablated to adjust correction of the refractive error in the same manner as described with respect to inlay 100. Coating 610 is then applied to inlay 600 and exposed corneal surface 608 adjacent inlay 600. More specifically, coating 610 is applied to front and side surfaces 620 and 622 of inlay and areas 626 of exposed cornea surface 608 adjacent inlay 600, as seen in FIG. 86. Coating 610 can also be applied to back surface 622. Coating 610 has adhesive or bonding properties to immobilize and bond inlay 600 to exposed corneal surface 608. Coating 610 can be any organic polymer or compound with bonding properties such as, fibronectin, collagen, vitronectin, or polysaccande. Coating 610 is applied in liquid form on inlay 600, as seen in FIG. 85. Any excess coating can be absorbed by a sponge.

Coating 610 is then dried for a short period of time, such as 1 second-5 minutes, crosslinking coating 610 to form a drape over inlay 600 and areas 626 of cornea surface 626, as seen in FIG. 86, thereby immobilizing inlay 600 in proper position on corneal surface 608. Coating 610 may be dried by exposure to ultraviolet light or air 616. Once dried, coating 610 may have a thickness of 0.01-5 microns.

Flap 606 is then replaced over inlay 600 and exposed cornea surface 608 with inlay 600 being immobilized in proper position. By not removing flap 606, flap 606 is preserved and growth of a new flap is not required.

Alternatively, coating 610 can be a membrane 630 that substantially encloses all or part of inlay 600, as seen in FIG. 88. Membrane 630 may be pre-formed prior to implantation. Membrane 630 may be formed of amniotic material.

While preferred embodiments have been chosen to illustrate the invention, it will be understood by those skilled in the art that various changes and modifications can be made therein without departing from the scope of the invention as defined in the appended claims.

1. A method of treatment of refractive errors of an eye, the eye including a central visual axis and a cornea with a first corneal layer overlying a second corneal layer, comprising the steps of:
separating a first surface of the first corneal layer from a second surface of the second corneal layer, thereby forming a flap and exposing the second surface;
implanting on the second surface an inlay adapted to correct a refractive error of the eye;
coating a surface of the inlay with a compound that promotes bonding with the cornea; and
replacing the flap over the inlay.

2. A method according to claim 1, wherein
the coating step takes place before the implanting step.

3. A method according to claim 1, wherein
the coating step takes place after the implanting step.

4. A method according to claim 1, wherein
the first corneal layer is the epithelium.

5. A method according to claim 1, wherein
the second corneal layer is the stroma.

6. A method according to claim 1, further comprising the step of:
drying the compound coating on the surface of the inlay and thereby forming a drape on the inlay.

7. A method according to claim 4, wherein
the drying step comprises applying ultraviolet light to the compound and crosslinking the compound.

8. A method according to claim 1, further comprising the step of
coating the exposed second surface adjacent the inlay with the compound for bonding the inlay to the exposed second surface of the second corneal layer.

9. A method according to claim 8, further comprising the step of
drying the compound coating on the exposed second surface and thereby forming a drape on the inlay and bonding the inlay to the exposed second surface.

10. A method according to claim 9, wherein
the drying step comprises applying ultraviolet light to the compound.

11. A method according to claim 1, wherein
the compound is an organic polymer.

12. A method according to claim 11, wherein
the compound is formed of one of the group consisting of fibronectin, collagen, vitronectin, and polysaccande.

13. A method according to claim 1, further comprising the step of:
ablating the inlay prior to coating the surface of the inlay with the compound.

14. A method according to claim 1, wherein
the inlay is organic.

15. A method according to claim 14, wherein
the inlay is formed of one of the group consisting of laminin, collagen, and vitronectin.

16. A method according to claim 1, wherein
the inlay is synthetic.

17. A method according to claim 16, wherein
the inlay is formed of one of the group consisting of silicone, hydrogel and hilafilcon.

18. A method according to claim 1, wherein
the inlay is a mixture of organic and synthetic materials.

19. A method according to claim 1, wherein
the coating step comprises substantially enclosing the inlay.

20. A method according to claim 19, wherein
the coating step comprises substantially enclosing the inlay in a membrane.

21. A method according to claim 20, wherein
the membrane is made of amniotic material.

22. A method according to claim 1, wherein
the inlay is formed using diffractive technology.

23. A method according to claim 1, wherein
the coating step comprises coating a second surface of the inlay.

24. A method according to claim 23, wherein
the coating step comprises coating a third surface of the inlay.

25. A method of treatment of refractive errors of an eye, the eye including a central visual axis and a cornea with a first corneal layer overlying a second corneal layer, comprising the steps of:
separating a first surface of the first corneal layer from a second surface of the second corneal layer, thereby exposing the second surface;
implanting on the second surface an inlay adapted to correct a refractive error of the eye;
coating a surface of the inlay after implanting the inlay with a compound that promotes bonding with the cornea;
coating the exposed second surface adjacent the inlay with the compound; and
drying the compound coating the inlay and the exposed second surface, thereby forming a drape over the inlay and bonding the inlay to the second surface.

26. A method according to claim 25, further comprising the step of
replacing the first surface of the first corneal layer over the inlay and the second surface of the second corneal layer.

27. A method according to claim 25, wherein
the drying step comprises applying ultraviolet light to the compound.

28. A method according to claim 25, wherein
the first corneal layer is the epithelium.

29. A method according to claim 25, wherein
the second corneal layer is the stroma.
